(19) United States
(12) Patent Application Publication
(10) Pub. No.: US 2020/0158737 A1
(43) Pub. Date: May 21, 2020

(54) METHODS OF MEASURING UBIQUITIN-LIKE MODIFICATIONS

(71) Applicant: Regents of the University of Minnesota, Minneapolis, MN (US)

(72) Inventor: Yue Chen, Minneapolis, MN (US)

(73) Assignee: Regents of the University of Minnesota, Minneapolis, MN (US)

(21) Appl. No.: 16/688,235

(22) Filed: Nov. 19, 2019

Related U.S. Application Data
(60) Provisional application No. 62/770,379, filed on Nov. 21, 2018.

(51) Int. Cl. G01N 33/68; C07K 1/107; C07C 279/12

(52) U.S. Cl. CPC G01N 33/6848; C07K 1/1077; C07C 279/12; G01N 2800/24; G01N 2800/382; G01N 2800/2814; G01N 2800/10

(57) ABSTRACT

Certain embodiments of the invention provide a method of quantifying ubiquitin-like modification in a test protein sample comprising:
a) contacting the test protein sample with a compound of formula (I) to provide a first labeled test protein sample;
b) contacting the first labeled test protein sample with a first enzyme, wherein the first enzyme cleaves the ubiquitin-like modification from all modified amino acid residues, to provide a second labeled test protein sample;
c) contacting the second labeled test protein sample with a compound of formula (II) to provide a third labeled test protein sample; and
d) measuring the molecular weight of the protein(s) in the third labeled test protein sample to quantify the ubiquitin-like modification in the test protein sample,
wherein the compound of formula I is isotopically labeled; the compound of formula II is isotopically labeled; or the compound of formula I and the compound of formula II are differentially isotopically labeled.

### CROSS-REFERENCE APPLICATION

This application claims priority to U.S. Provisional Application Ser. No. 62/770,379, filed Nov. 21, 2018. The entire content of the application referenced above is hereby incorporated by reference herein.

### GOVERNMENT FUNDING

This invention was made with government support under CHE-1753154 awarded by the National Science Foundation and under GM124896 awarded by the National Institutes of Health. The government has certain rights in the invention.

### BACKGROUND OF THE INVENTION

Ubiquitination (Ub) is an essential pathway in eukaryotic cells that controls the signaling flux in diverse biological processes (D. Komander, M. Rape, Annu Rev Biochem 2012, 81, 203-229; A. Ordureau, et al., Mol Cell 2015, 58, 660-676; Y. Zhang, Genes Dev 2003, 17, 2733-2740; J. Peng, et al., Nat Biotechnol 2003, 21, 921-926). The elegant and versatile E1-E2-E3 enzymatic cascade and the countering activities of deubiquitinases (DUBs) with diverse substrate and linkage specificities determine the physiological stoichiometry of ubiquitination at the site-specific level. This balance determines the overall fates of the substrate proteins as well as the physiological activities of associated signaling networks.

Advances in the quantitative proteomics, such as SILAC and isobaric tagging (S. E. Ong, et al., Mol Cell Proteomics 2002, 1, 376-386; Y. Oda, et al., Proc Natl Acad Sci USA 1999, 96, 6591-6596; P. L. Ross, et al., Mol Cell Proteomics 2004, 3, 1154-1169), have enabled system-wide discoveries of Ub dynamics during signaling processes (W. Kim, et al., Mol Cell 2011, 44, 325-340; S. A. Wagner, et al., Mol Cell Proteomics 2011, 10, M111 013284; N. D. Udeshi, et al., Mol Cell Proteomics 2013, 12, 825-831; A. E. Elia, et al., Mol Cell 2015, 59, 867-881; H. Tsuchiya, et al., Mol Cell 2017, 66, 488-502 e487; V. Akimov, et al., Nat Struct Mol Biol 2018, 25, 631-640). Although highly efficient, such quantitative analysis mainly focus on the relative quantifications of the modification with limited information on the endogenous abundance of ubiquitination. To overcome this limitation, absolute quantification of ubiquitination has been achieved using spike-in stable-isotope labeled synthetic peptide standards (UB-AQUA) (D. S. Kirkpatrick, et al., Nat Cell Biol 2006, 8, 700-710) or recombinant proteins (PSAQ) (S. E. Kaiser, et al., Nat Methods 2011, 8, 691-696). These absolute quantification strategies have been successfully applied to study the dynamics of polyubiquitin linkages in ER stress, DNA damage response, mitophagy and in vitro enzyme activities (D. S. Kirkpatrick, et al., Nat Cell Biol 2006, 8, 700-710; P. Xu, et al., Cell 2009, 137, 133-145; A. Ordureau, et al., Mol Cell 2018, 70, 211-227 e218; H. Mirzaei, et al., Mol Biosyst 2010, 6, 2004-2014).

Stoichiometry analysis is an emerging approach to quantify the fractional abundance of PTMs (A. Ordureau, et al., Mol Cell 2018, 70, 211-227 e218; C. M. Smith, et al., Anal Biochem 2003, 316, 23-33; Q. Zhang, et al., J Am Soc Mass Spectrom 2007, 18, 1569-1578; J. V. Olsen, et al., Sci Signal 2010, 3, ra3; J. Park, et al., Mol Cell 2013, 50, 919-930; K. L. Fiedler, R. J. Cotter, Anal Chem 2013, 85, 5827-5834; J. Baeza, et al., J Biol Chem 2014, 289, 21326-21338; E. S. Nakayasu, et al., Int J Proteomics 2014, 2014, 730725; B. T. Weinert, et al., Mol Syst Biol 2014, 10, 716; H. Huang, et al., Chem Rev 2015, 115, 2376-2418; C. Feller, et al., Mol Cell 2015, 57, 559-571; T. Zhou, et al., J Proteome Res 2016, 15, 1103-1113; J. G. Meyer, et al., J Am Soc Mass Spectrom 2016, 27, 1758-1771; B. T. Weinert, et al., Mol Cell Proteomics 2017, 16, 759-769; J. Gil, et al., J Biol Chem 2017, 292, 18129-18144; T. Zhou, et al., Oncotarget 2016, 7, 79154-79169). Compared to relative quantifications, stoichiometry analysis directly measures the prevalence of the modification and allows the quantitative comparison of PTM abundance between different sites on the same or different target proteins. Systematic analysis of PTM stoichiometries do not have to rely on the synthesis of in vitro isotopically labeled standards and therefore, potentially enables global untargeted discoveries of modification abundance at the physiological levels. Recent developments of new quantitative proteomics strategies have enabled stoichiometric analysis of phosphorylation, lysine acetylation, and succinylation on a global scale or in a target-specific manner (C. M. Smith, et al., Anal Biochem 2003, 316, 23-33; Q. Zhang, et al., J Am Soc Mass Spectrom 2007, 18, 1569-1578; J. V. Olsen, et al., Sci Signal 2010, 3, ra3; J. Baeza, et al., J Biol Chem 2014, 289, 21326-21338; E. S. Nakayasu, et al., Int J Proteomics 2014, 2014, 730725; B. T. Weinert, et al., Mol Syst Biol 2014, 10, 716; C. Feller, et al., Mol Cell 2015, 57, 559-571; T. Zhou, et al., J Proteome Res 2016, 15, 1103-1113; J. G. Meyer, et al., JAm Soc Mass Spectrom 2016, 27, 1758-1771; B. T. Weinert, et al., Mol Cell Proteomics 2017, 16, 759-769; J. Gil, et al., J Biol Chem 2017, 292, 18129-18144). Despite these advances, accurate and site-specific stoichiometric analysis of ubiquitination has been challenging.

Accordingly, new methods for analyzing ubiquitination and other ubiquitin-like modifications are needed.

### SUMMARY OF THE INVENTION

Described herein is the development of an efficient chemical-based quantitative proteomic approach (termed “IBAQ-Ub”), which may be used to quantify ubiquitin-like modification(s) (e.g., determine the absolute site-specific stoichiometry of ubiquitin-like modification(s)).

Accordingly, certain embodiments of the invention provide a method of quantifying ubiquitin-like modification in a test protein sample comprising:

a) contacting the test protein sample with a compound of formula (I):

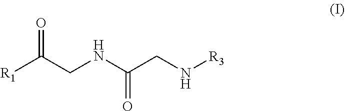

to provide a first labeled test protein sample, wherein R₁ together with the carbonyl group to which it is attached forms a group capable of reacting with an amino group to form an amide; and R₃ is X—Y—, wherein X is (C₁-C₆)alkanoyl and Y is absent or arginine;
b) contacting the first labeled test protein sample with a first enzyme, wherein the first enzyme cleaves ubiquitin-like protein(s) from all modified amino acid residues, to provide a second labeled test protein sample;

c) contacting the second labeled test protein sample with a compound of formula (II):

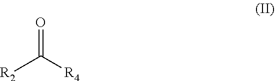

to provide a third labeled test protein sample, wherein R₂ together with the carbonyl group to which it is attached forms a group capable of reacting with an amino group to form an amide, and wherein R₄ is (C₁-C₅)alkyl; and

d) measuring the molecular weight of the protein(s) in the third labeled test protein sample to quantify the ubiquitin-like modification(s) in the test protein sample,

wherein the compound of formula I is isotopically labeled; the compound of formula II is isotopically labeled; or the compound of formula I and the compound of formula II are differentially isotopically labeled.

Certain embodiments of the invention provide a method of screening a test compound for modulating activity of ubiquitin-like modification, the method comprising:

a1) contacting a test protein sample with a test compound to provide a test protein reaction sample;

a2) contacting the test protein reaction sample with a compound of formula (I):

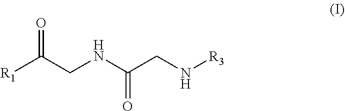

to provide a first labeled test protein reaction sample, wherein R₁ together with the carbonyl group to which it is attached forms a group capable of reacting with an amino group to form an amide; and R₃ is X—Y—, wherein X is (C₁-C₆)alkanoyl and Y is absent or arginine;

b) contacting the first labeled test protein reaction sample with a first enzyme, wherein the first enzyme cleaves ubiquitin-like proteins from all modified amino acid residues, to provide a second labeled test protein reaction sample;

c) contacting the second labeled test protein reaction sample with a compound of formula (II):

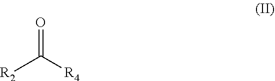

to provide a third labeled test protein reaction sample, wherein R₂ together with the carbonyl group to which it is attached forms a group capable of reacting with an amino group to form an amide, and wherein R₄ is (C₁-C₅)alkyl;

d) measuring the molecular weight of the protein(s) in the third labeled test protein reaction sample to quantify the ubiquitin-like modification(s) in the test protein sample,

wherein the compound of formula I is isotopically labeled; the compound of formula II is isotopically labeled; or the compound of formula I and the compound of formula II are differentially isotopically labeled; and

e) identifying the test compound as having modulating activity of ubiquitin-like modification when the amount or location of ubiquitin-like modification in the test protein sample is different than the ubiquitin-like modification in a corresponding control protein sample.

Certain embodiments of the invention provide a method of identifying a subject having a disease or disorder associated with altered ubiquitin-like modification, the method comprising:

a) contacting a test protein sample from the subject with a compound of formula (I):

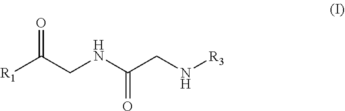

to provide a first labeled test protein sample, wherein R₁ together with the carbonyl group to which it is attached forms a group capable of reacting with an amino group to form an amide; and R₃ is X—Y—, wherein X is (C₁-C₆)alkanoyl and Y is absent or arginine;

b) contacting the first labeled test protein sample with a first enzyme, wherein the first enzyme cleaves ubiquitin-like protein(s) from all modified amino acid residues, to provide a second labeled test protein sample;

c) contacting the second labeled test protein sample with a compound of formula (II):

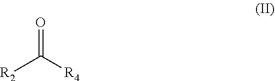

to provide a third labeled test protein sample, wherein R₂ together with the carbonyl group to which it is attached forms a group capable of reacting with an amino group to form an amide, and wherein R₄ is (C₁-C₅)alkyl;

d) measuring the molecular weight of the protein(s) in the third labeled test protein sample to quantify the ubiquitin-like modification(s) in the test protein sample,

wherein the compound of formula I is isotopically labeled; the compound of formula II is isotopically labeled; or the compound of formula I and the compound of formula II are differentially isotopically labeled; and

e) identifying the subject as having a disease or disorder associated with altered ubiquitin-like modification when the amount or location of modification(s) in the test protein sample is different than the modification(s) in a corresponding control protein sample.

Certain embodiments of the invention provide a kit for quantifying ubiquitin-like modification in a test protein sample, the kit comprising:

1) a compound of formula (I):

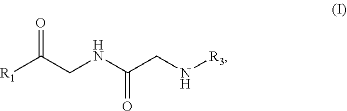

wherein R₁ together with the carbonyl group to which it is attached forms a group capable of reacting with an amino group to form an amide; and R₃ is X—Y—, wherein X is (C₁-C₆)alkanoyl and Y is absent or arginine;

2) a compound of formula (II):

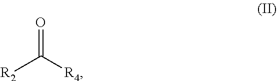

wherein R₂ together with the carbonyl group to which it is attached forms a group capable of reacting with an amino group to form an amide, and wherein R₄ is (C₁-C₅)alkyl; and

3) instructions for quantifying ubiquitin-like modification in the test protein sample,

wherein the compound of formula I is isotopically labeled; the compound of formula II is isotopically labeled; or the compound of formula I and the compound of formula II are differentially isotopically labeled.

Certain embodiments of the invention provide a compound of formula (Ia):

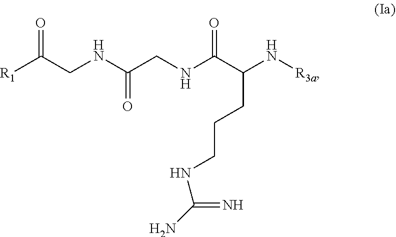

wherein R₁ is an activating group capable reacting with an amino group to form an amide; and R₃ₐ is (C₁-C₆)alkanoyl.

The invention also provides processes and intermediates disclosed herein that are useful for preparing a compound described herein or for use in a method described herein.

### BRIEF DESCRIPTION OF THE FIGURES

FIGS. 1A-1C. A chemical proteomics workflow for quantitative analysis of ubiquitination stoichiometry. (FIG. 1A) A ubiquitination site marked by a glycylglycine remnant after trypsin digestion. (FIG. 1B) The molecular structure of acetyl glycylglycine N-hydroxysuccinamide (AcGG-NHS) for ubiquitination stoichiometry analysis. (FIG. 1C) The IBAQ-Ub workflow that involves the derivatization of unmodified lysines with AcGG-NHS, trypsin digestion and stable isotopic labeling with heavy acetyl-NHS.

FIGS. 2A-2D. Stoichiometric analysis of the K48-linked di-Ub standard. (FIG. 2A) A schematic diagram for quantitative analysis of polyubiquitin linkage stoichiometry using IBAQ-Ub strategy. (FIG. 2B) Extracted ion chromatograms of K48 peptides with AcGG and heavy AcGG labeling. For each group, theoretical values are shown on the left and experimental values are shown on the right. (FIG. 2C) A representative mass spectrum to show the precursor ions of K48 peptides with AcGG and heavy AcGG labeling. (FIG. 2D) Stoichiometry quantification of ubiquitination linkages in K48-linked di-Ub standards. *Ac indicates heavy Ac.

FIGS. 3A-3C. Dynamic range of detection for the IBAQ-Ub workflow. (FIG. 3A) An experimental strategy for the measurement of the dynamic range of quantification for the IBAQ-Ub workflow. (FIG. 3B) A bar graph comparing the theoretical stoichiometry values in each sample with experimentally measured values. (FIG. 3C) A linear correlation line graph showing the correlation between theoretical stoichiometry values with the experimental measurements.

FIGS. 4A-4C. Analysis of the AcGG labeling efficiency in the complex cell lysate using a SILAC-based approach. (FIG. 4A) A SILAC-based strategy to measure the global site-specific labeling efficiency of AcGG-NHS in HT22 whole cell lysate. (FIG. 4B) Bar graphs and (FIG. 4C) Box and Whisker plots comparing the distributions of SILAC ratios between peptides that are theoretically affected by AcGG-NHS labeling on lysines and peptides that are not affected by AcGG-NHS labeling on lysines. In part (FIG. 4B), for each group the bar representing peptides that are theoretically affected by AcGG-NHS labeling on lysines are shown on the top and the bar representing peptides that are not affected by AcGG-NHS labeling on lysines are shown on the bottom. In part (FIG. 4C), the bar representing peptides that are theoretically affected by AcGG-NHS labeling on lysines is shown on the right and the bar representing peptides that are not affected by AcGG-NHS labeling on lysines is shown on the left.

FIGS. 5A-5C. Comparative analysis of ubiquitination stoichiometries in the histone enriched fractions of 293T cells treated with and without the proteasome inhibitor MG132. (FIG. 5A) A schematic diagram of the experimental workflow. (FIG. 5B) Bar graphs showing the labeling efficiencies of AcGG-NHS at the protein level across different whole cell lysate samples. (FIG. 5C) Bar graphs showing the labeling efficiencies of heavy acetyl-NHS at the peptide level across different samples.

FIGS. 6A-6B. Representative analysis of endogenous ubiquitination stoichiometries. (FIG. 6A) Stoichiometry analysis of histone H2B peptide “E.GTKAVTKYTSSK.-” (containing known K117 and K120 ubiquitination sites) based on the extracted ion chromatograms of AcGG (upper panels) and heavy AcGG (lower panels) labeled peptides under DMSO-treated (left panels) and MG132-treated samples. (FIG. 6B) Stoichiometry analysis of ubiquitin peptide “R.LIFAGKQLEDGR.T” (containing known K48 ubiquitination site) based on the extracted ion chromatograms of AcGG (upper panels) and heavy AcGG (lower panels) labeled peptides under DMSO-treated (left panels) and MG132-treated (right panels) samples.

FIG. 7. Schematic of IBAQ-Ub Analysis.

FIGS. 8A-8C. Synthesis and characterization of AcGG-NHS. (FIG. 8A) A schematic diagram for the synthesis of AcGG-NHS compound using acetylglycylglycine and N-hydroxysuccinamide as the starting material. (FIG. 8B) ¹H NMR and (FIG. 8C) electrospray (ESI) mass spectrum of the synthesized AcGG-NHS.

FIGS. 9A-9C. Ninhydrin test with bovine serum albumin (BSA) as standard to evaluate AcGG-NHS labeling efficiency. (FIG. 9A) The design of experiments to test the AcGG-NHS reactivity using BSA standard. (FIG. 9B) A representative picture and (FIG. 9C) bar graph showing a complete loss of Ninhydrin reactivity on BSA after AcGG-NHS labeling.

FIG. 10. The ubiquitination stoichiometries of known ubiquitination sites in the histone-enriched protein fractions that were quantified of both DMSO and MG-132 treated 293T cells. Within the figure, the following abbreviations are used: (ah): heavy acetyl labeling; (ac): AcGG labeling; and 0*: Not measurable due to the intensity of the corresponding heavy AcGG labeled peptide below the detection limit.

FIG. 11. Oxygen and metabolic-dependent regulation of Hypoxia-Inducible Factor 1 alpha (HIF1A).

FIG. 12. A schematic diagram illustrating the functional domains of HIF1A and the known ubiquitination sites by previous shot-gun proteomics analysis.

FIG. 13. Experimental workflow for stoichiometric quantification of ubiquitination regulation of HIF1A. Flag-tagged HIF1A was expressed in 293T cells. The cells were treated with 10 μM MG132 for 2 hr and 6 hr respectively. HIF1A was purified by immunoprecipitation with antibody against the Flag epitope. Proteins were labeled with AcGG-NHS and then resolved on SDS-PAGE. Following in-gel digestion, peptides were labeled with heavy acetyl NHS then analyzed by LCMS.

### DETAILED DESCRIPTION

### Certain Methods Described Herein

Certain embodiments of the invention provide a method of quantifying ubiquitin-like modification (e.g., ubiquitination, SUMOylation, ISGylation and/or neddylation) in a test protein sample comprising:

a) contacting the test protein sample with a compound of formula (I):

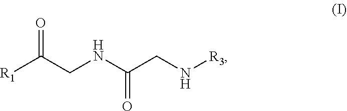

to provide a first labeled test protein sample, wherein R₁ together with the carbonyl group to which it is attached forms a group capable of reacting with an amino group to form an amide; and R₃ is X—Y—, wherein X is (C₁-C₆)alkanoyl and Y is absent or arginine;

b) contacting the first labeled test protein sample with a first enzyme, wherein the first enzyme cleaves the ubiquitin-like protein(s) from all modified amino acid residues (e.g., a lysine side chain), to provide a second labeled test protein sample;

c) contacting the second labeled test protein sample with an compound of formula (II):

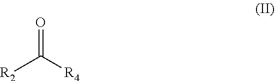

to provide a third labeled test protein sample, wherein R₂ together with the carbonyl group to which it is attached forms a group capable of reacting with an amino group to form an amide, and wherein R₄ is (C₁-C₅)alkyl;

and

d) measuring the molecular weight of the protein(s) in the third labeled test protein sample to quantify the ubiquitin-like modification(s) in the test protein sample,

wherein the compound of formula I is isotopically labeled; the compound of formula II is isotopically labeled; or the compound of formula I and the compound of formula II are differentially isotopically labeled.

Certain embodiments of the invention provide a method of screening a test compound for activity in modulating ubiquitin-like modification, the method comprising:

a1) contacting a test protein sample with a test compound to provide a test protein reaction sample;

a2) contacting the test protein reaction sample with a compound of formula (I):

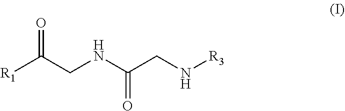

to provide a first labeled test protein reaction sample, wherein R₁ together with the carbonyl group to which it is attached forms a group capable of reacting with an amino group to form an amide; and R₃ is X—Y—, wherein X is (C₁-C₆)alkanoyl and Y is absent or arginine;

b) contacting the first labeled test protein reaction sample with a first enzyme, wherein the first enzyme cleaves the ubiquitin-like protein(s) from all modified amino-acid residues (e.g., a lysine side chain), to provide a second labeled test protein reaction sample;

c) contacting the second labeled test protein reaction sample with a compound of formula

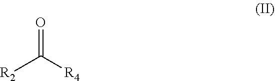

to provide a third labeled test protein reaction sample, wherein R₂ together with the carbonyl group to which it is attached forms a group capable of reacting with an amino group to form an amide, and wherein R₄ is (C₁-C₅)alkyl;

d) measuring the molecular weight of the protein(s) in the third labeled test protein reaction sample to quantify the ubiquitin-like modification(s) in the test protein sample,

wherein the compound of formula I is isotopically labeled; the compound of formula II is isotopically labeled; or the compound of formula I and the compound of formula II are differentially isotopically labeled; and

e) identifying the test compound as having activity in modulating ubiquitin-like modifications when the amount or location of the ubiquitin-like modification(s) in the test protein sample is different (e.g., more or less modifications or at different sites) than in a corresponding control protein sample (e.g., a negative control sample that was not contacted by the test compound).

As used herein, the phrase “activity in modulating ubiquitin-like modifications” refers to a compound that is capable of increasing or decreasing the amount and/or site of one or more ubiquitin-like modifications in a given protein or peptide.

Certain embodiments of the invention provide a method of identifying a subject having a disease or disorder associated with altered ubiquitin-like modification, the method comprising:

a) contacting a test protein sample from the subject with a compound of formula (I):

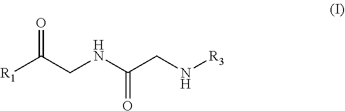

to provide a first labeled test protein sample, wherein R₁ together with the carbonyl group to which it is attached forms a group capable of reacting with an amino group to form an amide; and R₃ is X—Y—, wherein X is (C₁-C₆)alkanoyl and Y is absent or arginine;

b) contacting the first labeled test protein sample with a first enzyme, wherein the first enzyme cleaves ubiquitin-like protein(s) from all modified amino acid residues (e.g., lsine side chains), to provide a second labeled test protein sample;

c) contacting the second labeled test protein sample with a compound of formula (II):

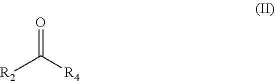

to provide a third labeled test protein sample, wherein R₂ together with the carbonyl group to which it is attached forms a group capable of reacting with an amino group to form an amide, and wherein R₄ is (C₁-C₅)alkyl;

d) measuring the molecular weight of the protein(s) in the third labeled test protein sample to quantify the ubiquitin-like modifications(s) in the test protein sample,

wherein the compound of formula I is isotopically labeled; the compound of formula II is isotopically labeled; or the compound of formula I and the compound of formula II are differentially isotopically labeled; and

e) identifying the subject as having a disease or disorder associated with altered ubiquitin-like modifications when the amount or location of the ubiquitin-like modification(s) in the test protein sample is different (e.g., more or less modifications or at different sites) than the modifications in a corresponding control protein sample (e.g., a sample from a subject that does not have the disease or disorder).

Diseases or disorders associated with altered ubiquitin-like modification (e.g., ubiquitination, SUMOylation, ISGylation and neddylation) are known in the art, and include but are not limited to, e.g., cancer, neurodegenerative diseases (e.g., Parkinson's disease, Alzheimer's disease), cystic fibrosis, muscle wasting and immunological disorders.

In certain embodiments, a method of the invention further comprises obtaining a protein sample from a subject (e.g., a mammal, such as a human). In certain embodiments, the protein sample is derived from a cell or tissue sample obtained from the subject.

In certain embodiments, the method further comprises administering a treatment to the subject identified with the disease or disorder. In certain embodiments, the effectiveness of a treatment may be evaluated using a method described herein (e.g., quantifying ubiquitin-like modification, such as ubiquitination, SUMOylation, ISGylation or neddylation) in a protein sample obtained from a patient before and after administration of the treatment).

Certain embodiments of the invention provide a method as described herein (e.g., as described in the Example).

### Ubiquitin-Like Modification

As described herein, a method of the invention may be used to quantify ubiquitin-like modification of a peptide or protein.

As used herein the term “ubiquitin-like modification” refers to ubiquitination and other post-translational modifications that are similar to ubiquitination, including but not limited to SUMOylation, ISGylation and neddylation. In certain embodiments, the modification comprises a protein, such as ubiquitin, small ubiquitin-like modifier (SUMO), interferon-stimulated gene 15 (ISG15) or neural-precursor-cell-expressed developmentally down-regulated 8 (NEDD8), which has a “GG” sequence at its C-terminus. For example, the amino acid sequence of ubiquitin terminates with an “RGG” sequence; the amino acid sequence of mature SUMO terminates with a “TGG” sequence; the amino acid sequence of ISG15 terminates with an “RGG” sequence; and the amino acid sequence of NEDD8 terminates with an “RGG” sequence. Thus, in certain embodiments, the ubiquitin-like modification is ubiquitination. In certain embodiments, the ubiquitin-like modification is SUMOylation. In certain embodiments, the ubiquitin-like modification is ISGylation. In certain embodiments, the ubiquitin-like modification is neddylation.

In certain embodiments, a method described herein is used to quantify ubiquitination. Ubiquitination refers to a post-translational modification of a substrate protein, wherein one or more ubiquitin proteins are added to the substrate protein. Ubiquitin may be bound to a lysine residue via an isopeptide bond; to a cysteine residue through a thioester bond; to a serine or threonine residue through an ester bond; or to the amino group of an N-terminal amino acid (e.g., methionine) via a peptide bond. In certain embodiments, a lysine residue is ubiquitinated. In certain embodiments, an N-terminus amino acid is ubiquitinated.

In certain embodiments, a method described herein is used to quantify SUMOylation. SUMOylation refers to a post-translational modification of a substrate protein, wherein one or more SUMO proteins are added to the substrate protein (e.g., SUMO may be bound to a lysine residue via an isopeptide bond).

In certain embodiments, a method described herein is used to quantify ISGylation. ISGylation refers to refers to a post-translational modification of a substrate protein, wherein one or more ISG15 proteins are added to the substrate protein (e.g., ISG15 may be bound to a lysine residue via an isopeptide bond).

In certain embodiments, a method described herein is used to quantify neddylation. Neddylation refers to a post-translational modification of a substrate protein, wherein one or more NEDD8 proteins are added to the substrate protein (e.g., NEDD8 may be bound to a lysine residue via an isopeptide bond).

### Protein Sample

As described herein, the term “protein sample” refers to a sample that comprises one or more proteins. In certain embodiments, the sample comprises a single protein (e.g., a single purified protein). In certain embodiments, the sample comprises a plurality of proteins (e.g., 2, 3, 4, 5, 6, 7, 8, 9, 10, 15, 20, 25, 30, 35, 40, 45, 50, 75, 100, 150, 200, 300, 400, 500 or more proteins).

In certain embodiments, the protein(s) is purified (e.g., from other cellular components). In certain embodiments, the protein(s) is present within cell lysate.

In certain embodiments, the protein sample is obtained from a subject (e.g., a mammal, such as a human). In certain embodiments, the protein sample is a cell sample or tissue sample. As described herein, the terms “quantifying ubiquitin-like modification in a protein sample” may refer to 1) measuring the total number of amino acid residues in the sample that are modified by ubiquitin or other ubiquitin-like protein, such as SUMO, ISG15 or NEDD8 (e.g., lysine residues or N-terminal residues); or 2) measuring the total number of amino acid residues that are modified by ubiquitin or other ubiquitin-like protein in one or more particular proteins present in the sample; 3) measuring the relative change in the modification (e.g., ubiquitination) abundance on specific sites; or 4) measuring the absolute stoichiometry of modifications (e.g., ubiquitination) on specific sites. In certain embodiments, this term may further refer to determining a particular site(s) of ubiquitination or other ubiquitin-like modification.

In certain embodiments, a protein of interest may be cleaved into two or more peptide fragments, wherein the modification (e.g., ubiquitination, SUMOylation, ISGylation or neddylation) of one or more of the peptide fragments is quantified as described herein.

In certain embodiments, the modification (e.g., ubiquitination, SUMOylation, ISGylation or neddylation) of at least one protein/peptide fragment in the protein sample is quantified. In certain embodiments, the modification of at least two proteins/peptide fragments in the protein sample are quantified. In certain embodiments, the modification of at least 3, 4, 5, 6, 7, 8, 9, 10, 11, 12, 13, 14, 15, 16, 17, 18, 19, 20, 25, 30 or more proteins/peptide fragments in the protein sample are quantified. In certain embodiments, the modification of all the proteins present in the sample are quantified.

In certain embodiments, the protein(s) present in the sample comprises one or more lysine residues. In certain embodiments, the protein(s) present in the sample comprises a plurality of lysine residues (e.g., 2, 3, 4, 5, 6, 7, 8, 9, 10, 15, 20, 25, 30, 35, 40, 45, 50, 75, 100 or more lysine residues).

In certain embodiments, one or more lysine residues are modified (e.g., ubiquitinated, SUMOylated, ISGylated and/or neddylated). In certain embodiments, a plurality of lysine residues are modified (e.g., 2, 3, 4, 5, 6, 7, 8, 9, 10, 15, 20, 25, 30, 35, 40, 45, 50, 75, 100 or more lysine residues). In certain embodiments, the N-terminal amino acid is modified (e.g., a methionine residue).

### Step A

As described herein, methods of the invention comprise the step of contacting a protein sample with a compound of formula (I):

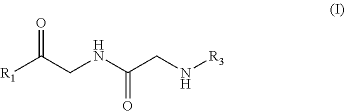

to provide a first labeled protein sample, wherein R₁ together with the carbonyl group to which it is attached forms a group capable of reacting with an amino group to form an amide (i.e., R₁ is an activating group capable reacting with an amino group to form an amide); and R₃ is X—Y—, wherein X is (C₁-C₆)alkanoyl and Y is absent or arginine.

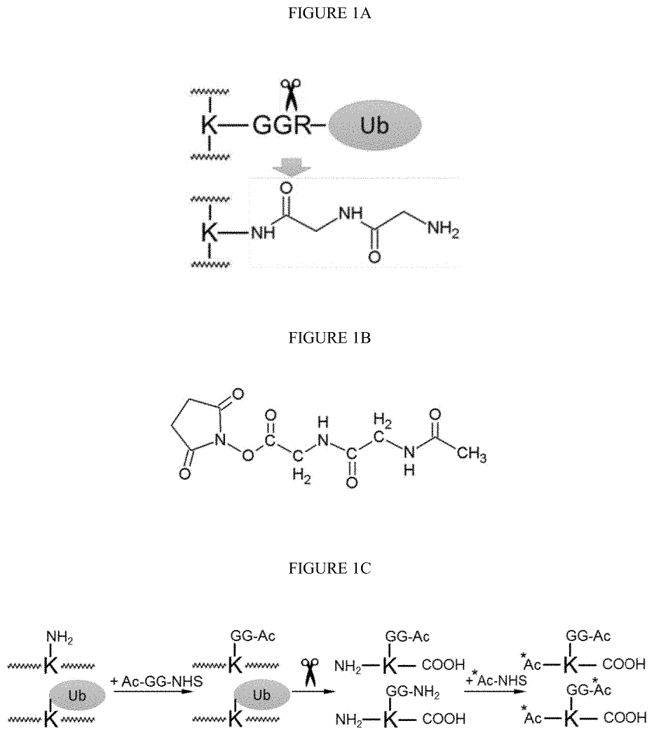

During such a contacting step as described in Step A, the activating group within the compound of formula (I) reacts with any free amines present in the protein sample, including but not limited to, non-ubiquitinated lysine side chains present in the protein(s). Thus, such a reaction results in labeled acyl-amines (e.g., acetylglyciclycyl (Ac-GG-) labeled lysine residue(s); see also, e.g., FIG. 1C). In certain embodiments, there are no free amines present in the protein sample. In certain embodiments, at least one protein present in the protein sample comprises at least one free amine. In certain embodiments, at least one protein present in the protein sample comprises at least two free amines. In certain embodiments, at least one protein present in the protein sample comprises, e.g., at least 3, 4, 5, 6, 7, 8, 9, 10, 11, 12, 13, 14, 15, 16, 17, 18, 19, 20, 25, 30 or more free amines.

As described herein, R₁ is an activating group capable reacting with an amino group to form an amide. In certain embodiments, R₁ has a molecular weight of less than about 500 amu. In certain embodiments, R₁ has a molecular weight of less than about 400 amu. In certain embodiments, R₁ has a molecular weight of less than about 300 amu. In certain embodiments, R₁ has a molecular weight of less than about 200 amu. In certain embodiments, R₁ has a molecular weight of less than about 150 amu. In certain embodiments, R₁ has a molecular weight of less than about 100 amu. In certain embodiments, R₁ has a molecular weight of less than about 50 amu. In certain embodiments, R₁ is:

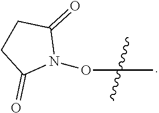 [SMILES: *ON1C(=O)CCC1=O]

In certain embodiments, R₁ is 2,3,5,6-tetrafluorophenol:

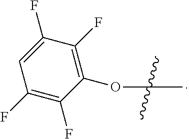 [SMILES: *Oc1c(F)c(F)cc(F)c1F]

As described herein, R₃ is X—Y—, wherein X is (C₁-C₆)alkanoyl and Y is absent or arginine.

In certain embodiments, X is C₁alkanoyl. In certain embodiments, X is C₂alkanoyl. In certain embodiments, X is C₃alkanoyl. In certain embodiments, X is C₄alkanoyl. In certain embodiments, X is C₅alkanoyl. In certain embodiments, X is C₆alkanoyl. In certain embodiments X is —C(═O)CH₃. In certain embodiments X is —C(═O)CH₂CH₃.

In certain embodiments, Y is absent. In certain embodiments, Y is arginine.

In certain embodiments, X is —C(═O)CH₃ and Y is absent. In certain embodiments, X is —C(═O)CH₃ and Y is arginine.

In certain embodiments of the invention, the compound of formula (I) is:

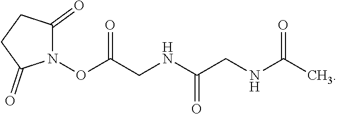 [SMILES: CC(=O)NCC(=O)NCC(=O)ON1C(=O)CCC1=O]

In certain embodiments of the invention, the compound of formula (I) is:

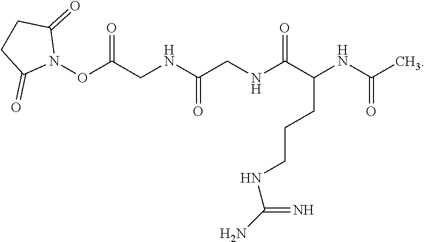 [SMILES: CC(=O)NC(CCCNC(=N)N)C(=O)NCC(=O)NCC(=O)ON1C(=O)CCC1=O]

In certain embodiments of the invention, the compound of formula (I) is:

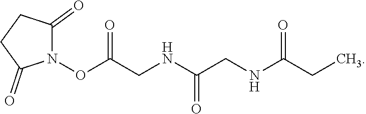 [SMILES: CCC(=O)NCC(=O)NCC(=O)ON1C(=O)CCC1=O]

In certain embodiments, a compound of formula I is isotopically labeled. Thus, in one embodiment, X is an isotopically labeled (C₁-C₆)alkanoyl. In certain embodiments, X is an isotopically labeled C₁alkanoyl. In certain embodiments, X is an isotopically labeled C₂alkanoyl. In certain embodiments, X is an isotopically labeled C₃alkanoyl. In certain embodiments, X is an isotopically labeled C₄alkanoyl. In certain embodiments, X is an isotopically labeled C₅alkanoyl. In certain embodiments, X is an isotopically labeled C₆alkanoyl. In certain embodiments X is an isotopically labeled —C(═O)CH₃. In certain embodiments X is an isotopically labeled —C(═O)CH₂CH₃.

The term “isotopically labeled” means enriched in at least one isotope above the natural abundance of that isotope at one or more positions of a compound. When a particular position, for example, a carbon atom, is isotopically labeled, it is understood that the abundance of one or more carbon isotopes (e.g. ¹³C) at that position is substantially greater than the natural abundance of that isotope (e.g. ¹³C), which for ¹³C is about 1.1%. An isotopically labeled position in a compound typically has a minimum isotopic enrichment factor of at least about 1000.

The term “isotopic enrichment factor” as used herein means the ratio between the isotopic abundance and the natural abundance of a specified isotope. In certain embodiments, a compound has an isotopic enrichment factor of at least 2500, or at least 4000.

Isotopes can be incorporated into a compound using a variety of known reagents and synthetic techniques.

In certain embodiments of the invention, the compound of formula (I) is an isotopically labeled compound of formula:

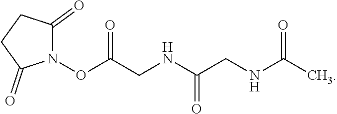 [SMILES: CC(=O)NCC(=O)NCC(=O)ON1C(=O)CCC1=O]

In certain embodiments of the invention, the compound of formula (I) is an isotopically labeled compound of formula:

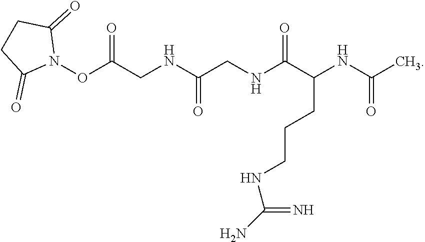 [SMILES: CC(=O)NC(CCCNC(=N)N)C(=O)NCC(=O)NCC(=O)ON1C(=O)CCC1=O]

In certain embodiments of the invention, the compound of formula (I) is an isotopically labeled compound of formula:

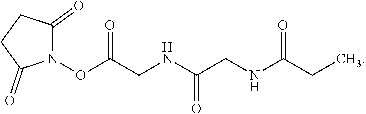 [SMILES: CCC(=O)NCC(=O)NCC(=O)ON1C(=O)CCC1=O]

In certain embodiments of the invention, the isotopically labeled compound of formula (I) is:

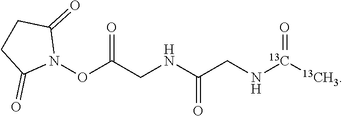

In certain embodiments of the invention, the isotopically labeled compound of formula (I) is:

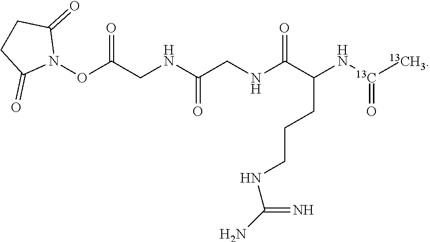

In certain embodiments of the invention, the isotopically labeled compound of formula (I) is:

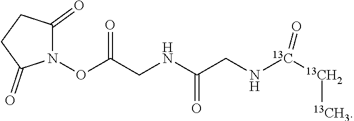

### Step B

As described herein, methods of the invention comprise the step of contacting the first labeled protein sample with a first enzyme, wherein the first enzyme cleaves ubiquitin-like modification(s) (e.g., ubiquitin, SUMO, ISG15 and/or NEDD8) from all modified amino acid residues (e.g., lysine side chains), to provide a second labeled protein sample.

In certain embodiments, the first labeled protein sample comprises no modified (e.g., ubiquitinated, SUMOylated, ISGylated or neddylated) amino acid residues (e.g., lysine side chains). In certain embodiments, at least one protein in the first labeled protein sample comprises at least one modified amino acid residue (e.g., lysine side chain). In certain embodiments, at least one protein in the first labeled protein sample comprises at least two modified amino acid residues (e.g., lysine side chains). In certain embodiments, at least one protein in the first labeled protein sample comprises, e.g., at least 3, 4, 5, 6, 7, 8, 9, 10, 11, 12, 13, 14, 15, 16, 17, 18, 19, 20, 25, 30 or more modified amino acid residues (e.g., lysine side chains).

The first enzyme may be any enzyme capable of cleaving ubiquitin-like protein(s) (e.g., ubiquitin, SUMO, ISG15, and/or NEDD8) from an amino acid residue (e.g. a lysine residue or an N-terminal residue, such as methionine), such that a remnant of the ubiquitin-like protein remains linked to the previously modified amino acid residue. Specifically, after the enzymatic cleavage, the previously modified amino acid residue is linked to -GG-NH₂. Thus, one skilled in the art may select the first enzyme based on the amino acid sequence of the ubiquitin-like protein and the cleavage recognition sequence of a given enzyme, such that the enzyme will cleave the ubiquitin-like protein and leave a -GG-NH₂ remnant attached to the substrate protein.

In certain embodiments, the first enzyme is trypsin. Trypsin is serine protease that cleaves, e.g., at the carboxyl side chain of arginine and lysine, except when either is followed by proline. For example, the ubiquitin protein has a RGG sequence at its C-terminal end. Thus, trypsin can cleave the ubiquitin protein between the arginine and glycine residues, leaving a -GG-NH₂ remnant attached to the previously ubiquitinated protein/peptide (e.g., attached to a lysine side chain present in the previously ubiquitinated protein/peptide). Similarly, the ISG15 and NEDD8 proteins also have a RGG sequence at its C-terminal end. Thus, trypsin can cleave the ISG15 or the NEDD8 protein between the arginine and glycine residues, leaving a -GG-NH₂ remnant attached to the previously ubiquitinated protein/peptide (e.g., attached to a lysine side chain present in the previously ubiquitinated protein/peptide).

In certain embodiments, the first enzyme is Arg-C. Arg-C, also known as clostripain, is an endopeptidase that cleaves at the carboxyl end of arginine residues, including the sites next to proline, and at carboxyl end of lysine residues. Thus, similar to trypsin, Arg-C will leave a -GG-NH₂ remnant attached to a previously ubiquitinated, ISGylated or neddylated amino acid residue (e.g., a lysine side chain) in a protein/peptide.

In certain embodiments, the first enzyme is an alpha-lytic protease, such as the wild-type alpha-lytic protease, WaLP. WaLP cleaves after T, A, S and V amino acid residues. For example, the SUMO protein has a TGG sequence at its C-terminal end. Thus, WaLP can cleave the SUMO protein between the threonine and glycine residues, leaving a -GG-NH₂ remnant attached to the previously SUMOylated protein/peptide (e.g., attached to a lysine side chain present in the previously SUMOylated protein/peptide).

In certain embodiments, the first enzyme further digests the protein(s) present in the sample into two or more peptide fragments (e.g., 2, 3, 4, 5, 6, 7, 8, 9, 10, 15, 20, 25, 30, 35, 40, 45, 50, 75, 100, 150, 200, 300, 400, 500 or more peptide fragments). In certain embodiments, the peptide fragments are between about 4 to about 50 amino acids in length. In certain embodiments, the peptide fragments are between about 4 to about 40 amino acids in length. In certain embodiments, the peptide fragments are between about 4 to about 30 amino acids in length. In certain embodiments, the peptide fragments are between about 4 to about 20 amino acids in length.

In certain embodiments, the peptide fragments are between about 4 to about 15 amino acids in length. In certain embodiments, the peptide fragments are between about 4, 5, 6, 7, 8, 9 or 10 amino acids in length.

In certain embodiments, the first enzyme digests one or more proteins in the first labeled test protein sample to provide a mixture of 2 or more peptide fragments in the second labeled protein sample.

### Step C

As described herein, methods of the invention comprise the step of contacting the second labeled protein sample with a compound of formula (II):

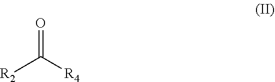

to provide a third labeled protein sample, wherein R₂ together with the carbonyl group to which it is attached forms a group capable of reacting with an amino group to form an amide (i.e., R₂ is an activating group capable reacting with an amino group to form an amide), and wherein R₄ is (C₁-C₅)alkyl. During such a contacting step, the activating group within the compound of formula (II) reacts with any free amines present in the second labeled protein sample, including but not limited to, the free amine group linked to any residues previously modified with a ubiquitin-like protein residues (e.g., a lysine labeled with -GG-NH₂). Thus, such a reaction results in labeled acyl-amines (e.g., labeled acetylglyciclycyl (Ac-GG-) labeled lysine residue(s); see also, e.g., FIG. 1C).

In certain embodiments, there are no free amines present in the second labeled protein sample. In certain embodiments, at least one protein present in the second labeled protein sample comprises at least one free amine. In certain embodiments, at least one protein present in the second labeled protein sample comprises at least two free amines. In certain embodiments, at least one protein present in the second labeled protein sample comprises, e.g., at least 3, 4, 5, 6, 7, 8, 9, 10, 11, 12, 13, 14, 15, 16, 17, 18, 19, 20, 25, 30 or more free amines.

As described herein, R₂ is an activating group capable reacting with an amino group to form an amide. In certain embodiments, R₂ has a molecular weight of less than about 500 amu. In certain embodiments, R₂ has a molecular weight of less than about 400 amu. In certain embodiments, R₂ has a molecular weight of less than about 300 amu. In certain embodiments, R₂ has a molecular weight of less than about 200 amu. In certain embodiments, R₂ has a molecular weight of less than about 150 amu. In certain embodiments, R₂ has a molecular weight of less than about 100 amu. In certain embodiments, R₂ has a molecular weight of less than about 50 amu.

In certain embodiments, R₂ is:

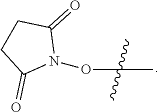 [SMILES: *ON1C(=O)CCC1=O]

In certain embodiments, R₂ is:

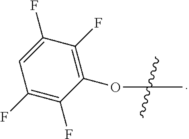 [SMILES: *Oc1c(F)c(F)cc(F)c1F]

In certain embodiments, R₄ is C₁alkyl. In certain embodiments, R₄ is C₂alkyl. In certain embodiments, R₄ is C₃alkyl. In certain embodiments, R₄ is C₄alkyl. In certain embodiments, R₄ is C₅alkyl.

In certain embodiments, the compound of formula (II) is:

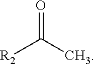

In certain embodiments, the compound of formula (II) is:

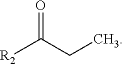

In certain embodiments, the compound of formula (II) is isotopically labeled.

In certain embodiments, the isotopically labeled compound of formula (II) is ¹³C₂D₃O—NHS. In certain embodiments, the isotopically labeled compound of formula (II) is D₃¹³C(═O)—NHS. In certain embodiments, the isotopically labeled compound of formula (II) is D₃ ¹³C¹³CD₂¹³C(═O)—NHS.

### Step D

As described herein, methods of the invention comprise the step of measuring the molecular weight of the protein(s) in the third labeled protein sample to quantify the ubiquitin-like modification (e.g., ubiquitination, SUMOylation, ISGylation and/or neddylation) in the protein sample.

As described herein, the terms “quantifying ubiquitin-like modification in a protein sample” may refer to 1) measuring the total number of amino acid residues in the sample that are modified by ubiquitin or other ubiquitin-like protein, such as SUMO, ISG15 or NEDD8 (e.g., lysine residues or N-terminal residues); 2) measuring the total number of amino acid residues that are modified by ubiquitin or other ubiquitin-like protein in one or more particular proteins present in the sample; 3) measuring the relative change in the modification (e.g., ubiquitination) abundance on specific sites; or 4) measuring the absolute stoichiometry of modifications (e.g., ubiquitination) on specific sites. In certain embodiments, this term may further refer to determining a particular site(s) of ubiquitination or other ubiquitin-like modification.

To enable quantification of ubiquitin-like modification, a compound of formula I, a compound of formula II or both compounds of formula I and II should be isotopically labeled. In certain embodiments, a compound of formula I is isotopically labeled. In certain embodiments, a compound of formula II is isotopically labeled. In certain embodiments, both a compound of formula I and a compound of formula II are differentially isotopically labeled. As used herein, the term “differentially isotopically labeled” refers to using either different isotopes and/or a different number of isotope labels in the compound of formula I and II.

A protein that is subjected to a method of the invention will have a higher molecular weight if the protein comprised one or more amino acid residues that were modified with ubiquitin-like protein(s) (e.g., lysine residues) as compared to a corresponding control protein that did not comprise modified amino acid residues. Similarly, a protein that is subjected to a method of the invention will have a lower molecular weight if the protein did not comprise one or more modified amino acid residues (e.g., ubiquitinated, SUMOylated, ISGylated and/or neddylated residues) as compared to a corresponding control protein that did comprise modified residues.

As used herein, the term “corresponding control protein” refers to a protein/peptide with a known modification status (e.g., ubiquitination, SUMOylation, ISGylation and/or neddylation status), which has the same amino acid sequence as the test protein/peptide. In certain embodiments, the corresponding control protein is a negative control, wherein the protein is not modified in a particular manner. For example, the corresponding negative control protein is not ubiquitinated, SUMOylated, ISGylated and/or neddylated.

In certain embodiments, the molecular weight of a non-modified corresponding control protein is measured using a method described herein. In certain embodiments, the molecular weight of a test protein and a corresponding control protein are compared to quantify ubiquitin-like modification of the test protein. Specifically, the difference in molecular weight between a test protein and a non-modified corresponding control protein, wherein both proteins have been subjected to a method of the invention, will be the molecular weight of the isotope label(s) multiplied by the number of modified residues present in the test protein. For example, if a test protein comprises two modified lysine residues, the difference in the molecular weight between the test protein and a non-modified corresponding control protein will be the molecular weight of the isotope label(s) multiplied by two.

In certain embodiments, a method described herein further comprises comparing the molecular weight of the protein(s) in the third labeled test protein sample to the molecular weight of a corresponding control protein(s), to quantify the ubiquitin-like modification(s) in the test protein sample.

In certain embodiments, the test protein sample comprises one or more protein(s) of interest (i.e., test proteins) and one or more corresponding control protein(s). Accordingly, in certain embodiments, a method described herein further comprises the step of measuring the molecular weight of a corresponding control protein(s) in the third labeled protein sample.

In certain embodiments, the corresponding control protein(s) is present in a separate sample (i.e., a corresponding control protein sample). Thus, in certain embodiments, the method further comprises:

e) contacting a corresponding control protein sample with a compound of formula (I):

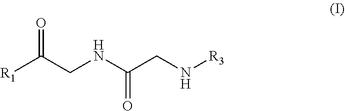

to provide a first labeled control protein sample, wherein R₁ together with the carbonyl group to which it is attached forms a group capable of reacting with an amino group to form an amide; and R₃ is X—Y—, wherein X is (C₁-C₆)alkanoyl and Y is absent or arginine;

f) contacting the first labeled control protein sample with the first enzyme, wherein the first enzyme cleaves ubiquitin-like proteins from all modified amino acid side chains, to provide a second labeled control protein sample;

g) contacting the second labeled control protein sample with a compound of formula (II):

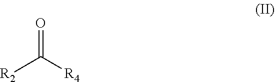

to provide a third labeled control protein sample, wherein R₂ together with the carbonyl group to which it is attached forms a group capable of reacting with an amino group to form an amide, and wherein R₄ is (C₁-C₅)alkyl; and

h) measuring the molecular weight of the protein(s) in the third labeled control protein sample.

In certain embodiments, the method further comprises comparing the molecular weight of the protein(s) in the third labeled test protein sample to the molecular weight of the protein(s) in the third labeled control protein sample (e.g., to quantify the ubiquitin-like modification(s) in the test protein sample).

In certain embodiments, the amino acid sequence of the test protein is known. As such, the molecular weight of the protein may be predicted, assuming no modification (e.g., ubiquitination, SUMOylation, ISGylation and/or neddylation) of any amino acid residues, partial modification or modification of all possible residues (i.e., all lysine and N-terminal residues). The predicted molecular weight may then be compared to the actual molecular weight of the test protein, thereby enabling quantification of the modification (e.g., ubiquitination, SUMOylation, ISGylation and/or neddylation) in the test protein sample.

In certain embodiments, the amount of ubiquitin-like modification (e.g., ubiquitination, SUMOylation, ISGylation and/or neddylation) is increased, e.g., increased by about 10%, 15%, 20%, 25%, 30%, 35%, 40%, 45%, 50%, 55%, 60%, 65%, 70%, 75%, 80%, 85%, 90%, 95%, 100% or more, as compared to that in a corresponding control protein sample. In certain embodiments, the amount of ubiquitin-like modification is decreased, e.g., decreased by about 10%, 15%, 20%, 25%, 30%, 35%, 40%, 45%, 50%, 55%, 60%, 65%, 70%, 75%, 80%, 85%, 90%, 95%, 100% or more, as compared to that in a corresponding control protein sample.

In certain embodiments, methods of the invention enable site-specific stoichiometry analysis of ubiquitin-like modification (e.g., when the test sample/protein comprises a single lysine residue; or when the test sample/protein comprises multiple lysine residues but all or none are determined to be modified by a ubiquitin-like protein).

In certain embodiments, the molecular weight of the protein(s) in the third labeled protein sample is measured using mass spectrometry. In certain embodiments, the molecular weight of the protein(s) in the third labeled protein sample is measure using Orbitrap, Quadrupole, Ion trap, Time-Of-Flight or FT-ICR mass spectrometers. In certain embodiments, the molecular weight of the protein(s) in the third labeled protein sample is measured using liquid chromatography-mass spectrometry (LC-MS).

### Second Digestion

In certain embodiments, the second labeled protein sample is contacted with a second enzyme to further digest the protein(s) present in the sample. This second digestion may be used to reduce the length of each protein/peptide present in the sample to facilitate the molecular weight analysis (e.g., by mass spectrometry). For example, in certain embodiments, the second digestion generates one or more peptides having a length of about 4 to about 30 amino acids in length (e.g., 4, 5, 6, 7, 8, 9, 10, 11, 12, 13, 14, 15, 16, 17, 18, 19, 20, 21, 22, 23, 24, 25, 26, 27, 28, 29 or 20 amino acids in length).

Thus, in certain embodiments, a method described herein, comprises contacting the second labeled test protein sample with a second enzyme, wherein the second enzyme digests one or more proteins in the second labeled test protein sample to provide a mixture of 2 or more peptide fragments.

The second enzyme may be any enzyme that is capable of further digesting the protein(s) present in the sample (i.e., different from the first enzyme). In certain embodiments, the second enzyme does not cut at the carboxyl side chain of an arginine, lysine, threonine, alanine, serine and/or valine residue. In certain embodiments, the second enzyme does not cut at the carboxyl side chain of an arginine residue. In certain embodiments, the second enzyme does not cut at the carboxyl side chain of a lysine residue. In certain embodiments, the second enzyme does not cut at the carboxyl side chain of a threonine residue. In certain embodiments, the second enzyme is not trypsin. In certain embodiments, the second enzyme is not Arg-C. In certain embodiments, the second enzyme is not WaLP.

In certain embodiments, the second enzyme is Glu-C, Asp-N, chymotrypsin, pepsin, aminopeptidase, carboxypeptidase, elastase, thermolysin or TEV protease. In certain embodiments, the second enzyme is Glu-C. In certain embodiments, the second enzyme is Asp-N.

### Kits

The present invention further provides kits for practicing the present methods.

Accordingly, certain embodiments of the invention provide a kit for quantifying ubiquitin-like modification (e.g., ubiquitination, SUMOylation, ISGylation and/or neddylation) in a test protein sample, the kit comprising:

1) a compound of formula (I) as described herein;

2) an isotopically labeled compound of formula (II) as described herein; and

3) instructions for use.

Certain embodiments of the invention provide a kit for quantifying ubiquitin-like modification (e.g., ubiquitination, SUMOylation, ISGylation and/or neddylation) in a test protein sample, the kit comprising:

1) an isotopically labeled compound of formula (I) as described herein;

2) a compound of formula (II) as described herein; and

3) instructions for use.

Certain embodiments of the invention provide a kit for quantifying ubiquitin-like modification (e.g., ubiquitination, SUMOylation, ISGylation and/or neddylation) in a test protein sample, the kit comprising:

1) an isotopically labeled compound of formula (I) as described herein;

2) an isotopically labeled compound of formula (II) as described herein; and

3) instructions for use

wherein, the compound of formula (I) and the compound of formula (II) are differentially isotopically labeled.

In certain embodiments, the compound of formula (I) is:

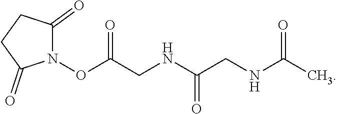 [SMILES: CC(=O)NCC(=O)NCC(=O)ON1C(=O)CCC1=O]

In certain embodiments, the compound of formula (II) is an isotopically labeled compound of formula (II): ¹³C₂D₃O—NHS.

In certain embodiments, the kit may further comprise a first enzyme as described herein (e.g., trypsin, Asp-N and/or Glu-C) and/or a second enzyme as described herein. Such kits may optionally contain one or more of: a positive and/or negative control, laboratory plastic ware and one or more buffers.

### Certain Compounds Described Herein

Certain embodiments of the invention provide a compound of formula (Ia):

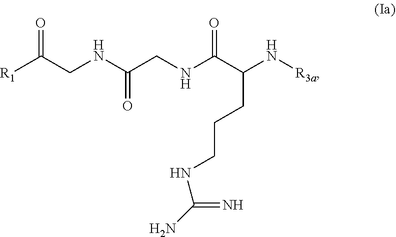

wherein R₁ is an activating group capable reacting with an amino group to form an amide; and R₃ₐ is (C₁-C₆)alkanoyl.

In certain embodiments, R₁ has a molecular weight of less than about 500 amu. In certain embodiments, R₁ has a molecular weight of less than about 400 amu. In certain embodiments, R₁ has a molecular weight of less than about 300 amu. In certain embodiments, R₁ has a molecular weight of less than about 200 amu. In certain embodiments, R₁ has a molecular weight of less than about 150 amu. In certain embodiments, R₁ has a molecular weight of less than about 100 amu. In certain embodiments, R₁ has a molecular weight of less than about 50 amu. In certain embodiments, R₁ is:

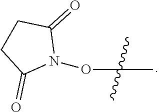 [SMILES: *ON1C(=O)CCC1=O]

In certain embodiments, R₁ is 2,3,5,6-tetrafluorophenol:

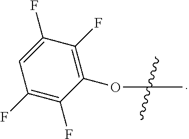 [SMILES: *Oc1c(F)c(F)cc(F)c1F]

In certain embodiments, R₃ₐ is C₁alkanoyl. In certain embodiments, R₃ₐ is C₂alkanoyl. In certain embodiments, R₃ₐ is C₃alkanoyl. In certain embodiments, R₃ₐ is C₄alkanoyl. In certain embodiments, R₃ₐ is C₅alkanoyl. In certain embodiments, R₃ₐ is C₆alkanoyl. In certain embodiments R₃ₐ is —C(═O)CH₃. In certain embodiments R₃ₐ is —C(═O)CH₂CH₃.

In certain embodiments, the compound of formula (Ia) is:

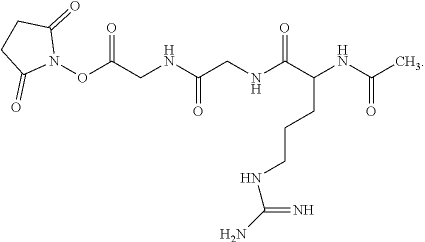 [SMILES: CC(=O)NC(CCCNC(=N)N)C(=O)NCC(=O)NCC(=O)ON1C(=O)CCC1=O]

In certain embodiments of the invention, the compound of formula (Ia) is isotopically labeled. Thus, in one embodiment, R₃ₐ is an isotopically labeled (C₁-C₆)alkanoyl. In certain embodiments, R₃ₐ is an isotopically labeled C₁alkanoyl. In certain embodiments, R₃ₐ is an isotopically labeled C₂alkanoyl. In certain embodiments, R₃ₐ is an isotopically labeled C₃alkanoyl. In certain embodiments, R₃ₐ is an isotopically labeled C₄alkanoyl. In certain embodiments, R₃ₐ is an isotopically labeled C₅alkanoyl. In certain embodiments, R₃ₐ is an isotopically labeled C₆alkanoyl. In certain embodiments R₃ₐ is an isotopically labeled —C(═O)CH₃. In certain embodiments R₃ₐ is an isotopically labeled —C(═O)CH₂CH₃.

In certain embodiments of the invention, the compound of formula (Ia) is an isotopically labeled compound of formula:

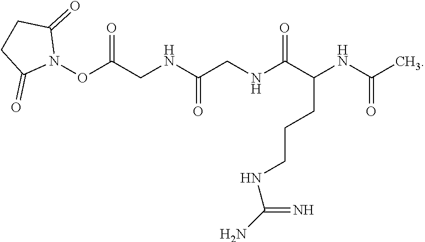 [SMILES: CC(=O)NC(CCCNC(=N)N)C(=O)NCC(=O)NCC(=O)ON1C(=O)CCC1=O]

In certain embodiments of the invention, the isotopically labeled compound of formula (Ia) is:

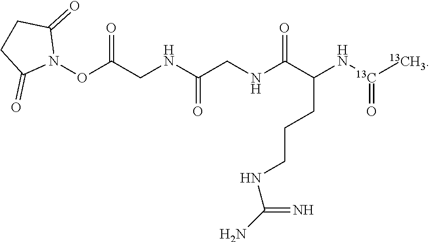

Certain embodiments of the invention provide a composition comprising a compound of formula (Ia) and a carrier.

### Certain Definitions

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. Dap, PyrAla, ThiAla, (pCl)Phe, (pNO₂)Phe, ε-Aminocaproic acid, Met[O₂], dehydPro, (3I)Tyr, norleucine (Nle), para-I-phenylalanine ((pI)Phe), 2-napthylalanine (2-NaI), β-cyclohexylalanine (Cha), β-alanine (β-Ala), phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid (Tic), penicillamine, ornithine, citruline, α-methyl-alanine, para-benzoylphenylalanine, phenylglycine, propargylglycine, sarcosine, and tert-butylglycine) in D or L form. The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein). An amino acid can be linked to the remainder of a compound of formula I through the carboxy terminus, the amino terminus, or through any other convenient point of attachment, such as, for example, through the sulfur of cysteine.

The term “peptide” describes a sequence of 2 to 25 amino acids (e.g. as defined hereinabove) or peptidyl residues. Peptide derivatives can be prepared as disclosed in U.S. Pat. Nos. 4,612,302; 4,853,371; and 4,684,620, or as described in the Examples hereinbelow. Peptide sequences specifically recited herein are written with the amino terminus on the left and the carboxy terminus on the right. The terms “protein,” “peptide” and “polypeptide” are used interchangeably herein.

The following terms are used to describe the sequence relationships between two or more sequences (e.g., polypeptides): (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length peptide sequence or the complete peptide sequence.

(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988) CABIOS, 4:11; the local homology algorithm of Smith et al. (1981) Adv. Appl. Math. 2:482; the homology alignment algorithm of Needleman and Wunsch, (1970) JMB, 48:443; the search-for-similarity-method of Pearson and Lipman, (1988) Proc. Natl. Acad. Sci. USA, 85:2444; the algorithm of Karlin and Altschul, (1990) Proc. Natl. Acad. Sci. USA, 87:2264, modified as in Karlin and Altschul, (1993) Proc. Natl. Acad. Sci. USA, 90:5873.

Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237; Higgins et al. (1989) CABIOS 5:151; Corpet et al. (1988) Nucl. Acids Res. 16:10881; Huang et al. (1992) CABIOS 8:155; and Pearson et al. (1994) Meth. Mol. Biol. 24:307. The ALIGN program is based on the algorithm of Myers and Miller, supra. The BLAST programs of Altschul et al. (1990) JMB, 215:403; Nucl. Acids Res., 25:3389 (1990), are based on the algorithm of Karlin and Altschul supra.

Software for performing BLAST analyses is publicly available through the National Center for Biotechnology Information (available on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of sequences for determination of percent sequence identity to another sequence may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

The term “subject” as used herein refers to humans, higher non-human primates, rodents, domestic, cows, horses, pigs, sheep, dogs and cats. In certain embodiments, the subject is a human.

The terms “treat” and “treatment” refer to both therapeutic treatment and prophylactic or preventative measures, wherein the object is to prevent or decrease an undesired physiological change or disorder, such as the development of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The invention will now be illustrated by the following non-limiting Examples.

### Example 1. A Quantitative Chemical Proteomics Approach for Site-Specific Stoichiometry Analysis of Ubiquitination

Stoichiometric analysis of posttranslational modifications is an emerging strategy for absolute quantification of the modification's fractional abundance. Here, a quantitative chemical proteomic workflow is reported for stoichiometric analysis of ubiquitination, named Isotopically BAlanced Quantification of Ubiquitination (IBAQ-Ub). The strategy utilizes a new amine-reactive chemical tag (AcGG-NHS) that is a structural homolog to the GG remnant of ubiquitin on modified lysine after trypsin cleavage and therefore enables the generation of structurally identical peptides from ubiquitinated and unmodified lysine residues following trypsin digestion and a secondary stable isotopic labeling. As shown herein, the strategy is highly robust, sensitive and accurate with a wide dynamic range using either protein standards or complex cell lysates. Thus, this work provides an efficient chemical proteomics tool for quantitative stoichiometric analysis of ubiquitination signaling pathways.

An Isotopically BAlanced Quantification strategy was designed for stoichiometry analysis of ubiquitination based on the chemical signature of the glycylglycine (GG) remnant on Ub sites after trypsin digestion (FIG. 1A). The major challenge of this workflow is to identify an effective strategy that enables the derivatization of unmodified lysine with an isotopically labeled GG remnant. To overcome this challenge, an acetyl glycylglycine (AcGG) tag that is structurally similar to the GG remnant was developed (FIG. 1B). The N-terminal acetylation of AcGG tag allowed the tag to be activated through NHS esterification by protecting the tag from self-conjugation. This way, unmodified lysines can be completely labeled with an AcGG tag (FIG. 1C). In the next step, trypsin digestion will cleave the ubiquitin and generate a GG remnant on originally ubiquitinated lysine with a fresh N-terminus. The second labeling with heavy acetyl-NHS (¹³C₂D₃-NHS) can then be applied, which serves two purposes. First, it balances the chemical structure of previously ubiquitinated lysine (with GG remnant) and previously unmodified lysine (with AcGG tag). Second, it incorporates stable isotopes only to the lysine with GG remnant and therefore, differentiates previously ubiquitinated lysines from previously unmodified lysines. Following these steps, originally ubiquitinated peptides and their original unmodified counterparts will have exactly the same chemical structure with the only difference in stable isotope labeling (FIG. 1C). Therefore, these peptides can be analyzed by routine LCMS for direct stoichiometry quantification based on the MS intensities.

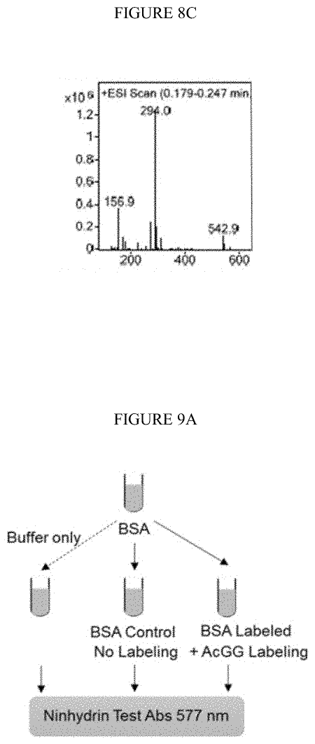

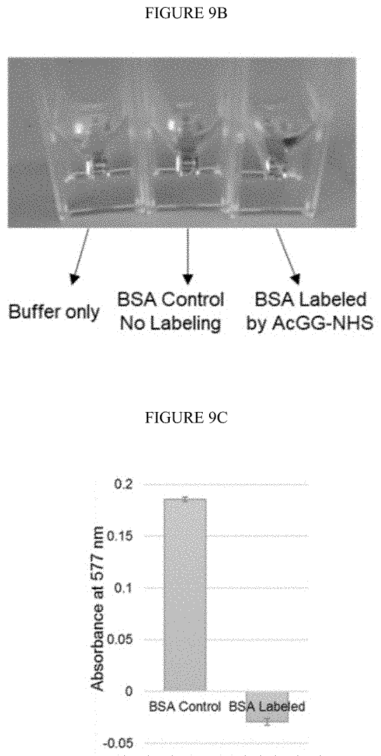

To validate this workflow, the AcGG-NHS and heavy acetyl-NHS were first synthesized (FIGS. 8A-C). The reactivity of acetyl-NHS to label lysine residues has been previously demonstrated (T. Zhou, et al., J Proteome Res 2016, 15, 1103-1113). Using Ninhydrin test with bovine serum albumin (BSA) standard, it was further demonstrated that AcGG-NHS has a high reactivity to derivatize amino groups (FIGS. 9A-C).

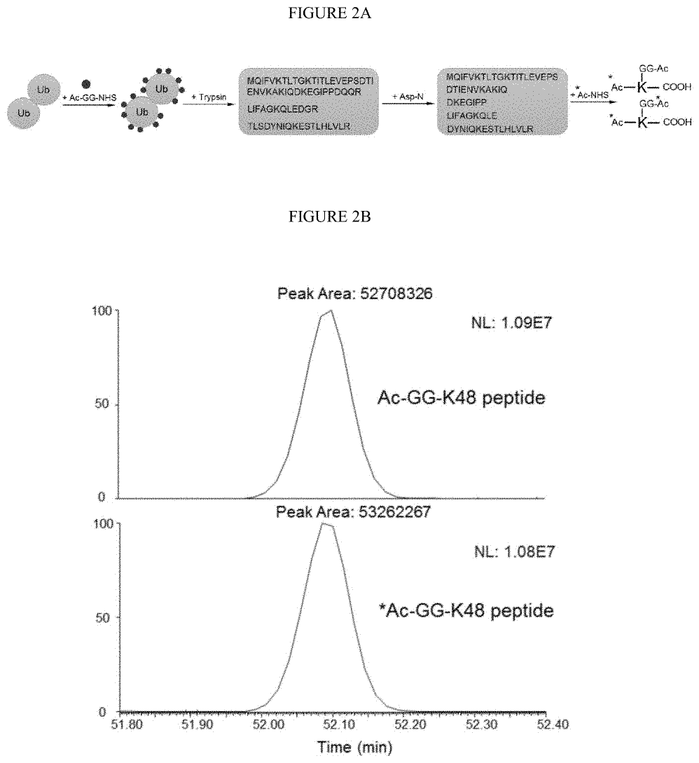

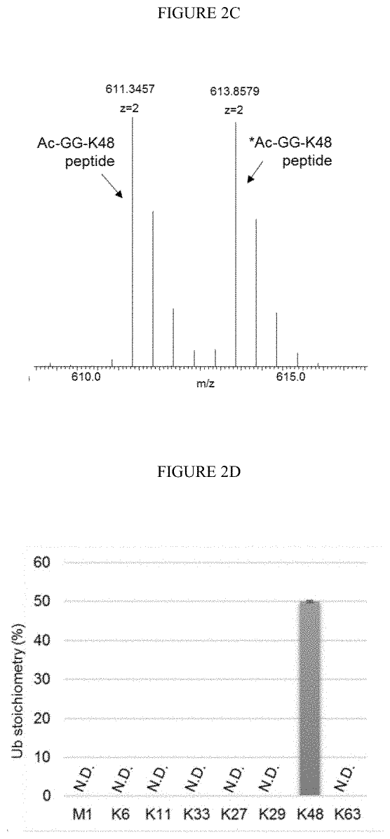

Next, it was explored if the IBAQ-Ub strategy would allow an accurate determination of Ub stoichiometry. To this end, the strategy was applied to analyze the recombinant di-Ub standard with K48 linkage (FIGS. 2A-D). To generate ubiquitin peptides with suitable lengths for LCMS analysis, a second digestion with a different protease such as Asp-N was included (FIG. 2A). The results showed that IBAQ-Ub analysis precisely determined the absolute stoichiometry of K48-linked di-Ub standard with a measurement of 50.14%±0.24% (FIG. 2B-2D).

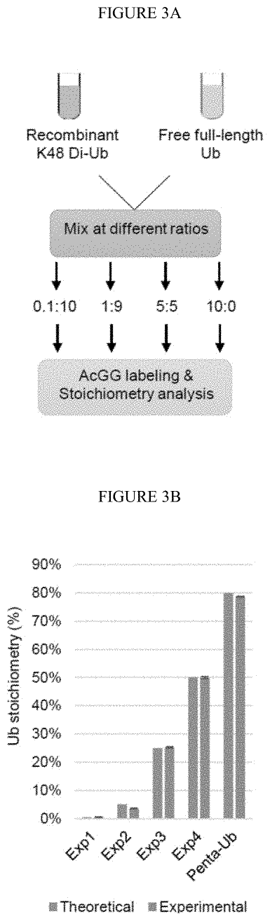

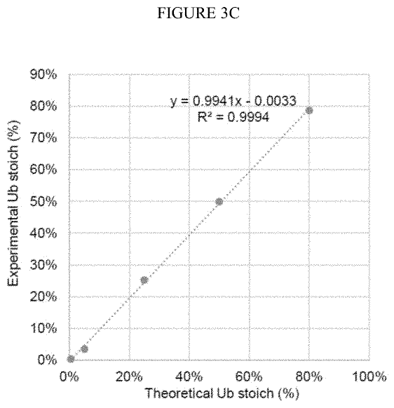

To determine the dynamic range and sensitivity of IBAQ-Ub workflow, a serial dilution experiment was performed by mixing recombinant K48-linked di-Ub with recombinant free ubiquitin at various ratios. These mixture would give a wide range of theoretical Ub stoichiometries from 0.5% to 50% (FIG. 3A). Each mixture was analyzed using IBAQ-Ub workflow independently with replicates. To determine if the strategy could measure longer polyubiquitin chains, the commercially available penta-Ub protein with K48 linkage was obtained. To ensure the purity of the penta-Ub standard, SDS-PAGE-based purification was applied. Analysis of di-Ub mixture and penta-Ub standard with multiple replicates showed that the experimental measurements of Ub stoichiometry were highly accurate and reproducible when comparing to theoretically expected values with a linear correlation coefficient R² of 0.9994 (FIG. 3B-3C), suggesting that IBAQ-Ub workflow is highly sensitive and capable of measuring a wide range of Ub stoichiometries with high accuracy.

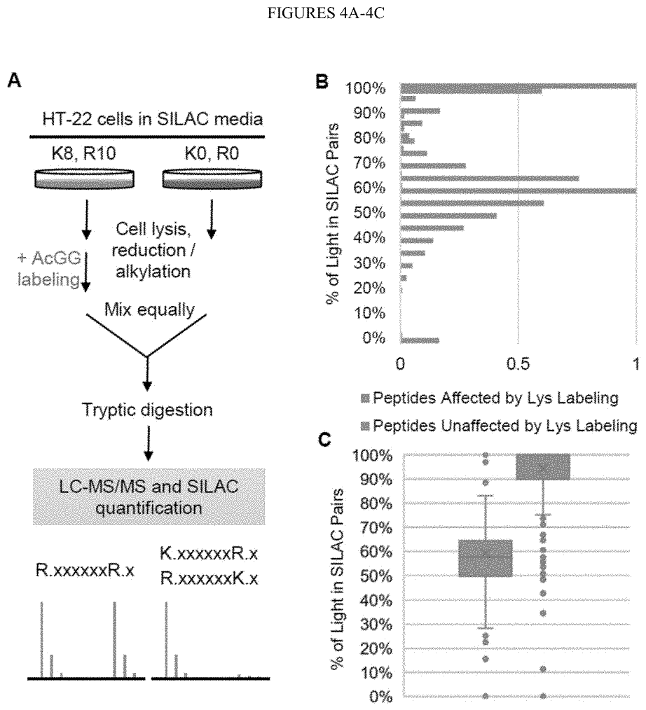

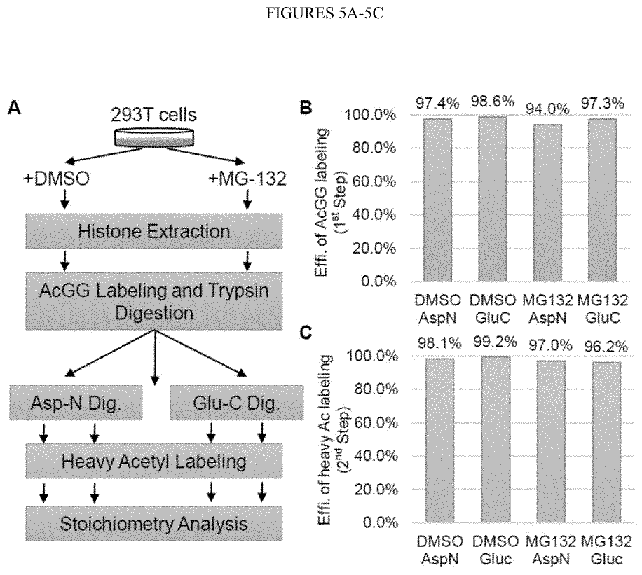

To determine if the IBAQ-Ub workflow is suitable for the analysis of a complex protein mixture, the site-specific labeling efficiency of the strategy was estimated using a SILAC-based approach (FIG. 4A), where proteins from heavy SILAC-labeled HT22 cells were subject to AcGG-NHS labeling before mixing with light-labeled proteins for quantitative analysis (FIG. 4A). By comparing the SILAC ratios of peptides affected and not affected by AcGG labeling, it was found that derivatization with AcGG-NHS achieved more than 90% site-specific labeling efficiency overall in a complex cell lysate (FIG. 4B-4C).

The IBAQ-Ub workflow was applied for site-specific stoichiometric analysis of known Ub sites on acid-extracted histones from 293T cells treated with DMSO (as control) or MG132 (a proteasome inhibitor) (FIG. 5A). To ensure the reliability of the analysis, the labeling efficiencies were checked based on LCMS data. The data showed that the labeling efficiencies of both AcGG-NHS and heavy Ac—NHS were very high and consistent across different samples (FIG. 5B-5C), suggesting that IBAQ-Ub workflow is highly robust for complex protein mixtures including proteins like histones with a large number of lysines.

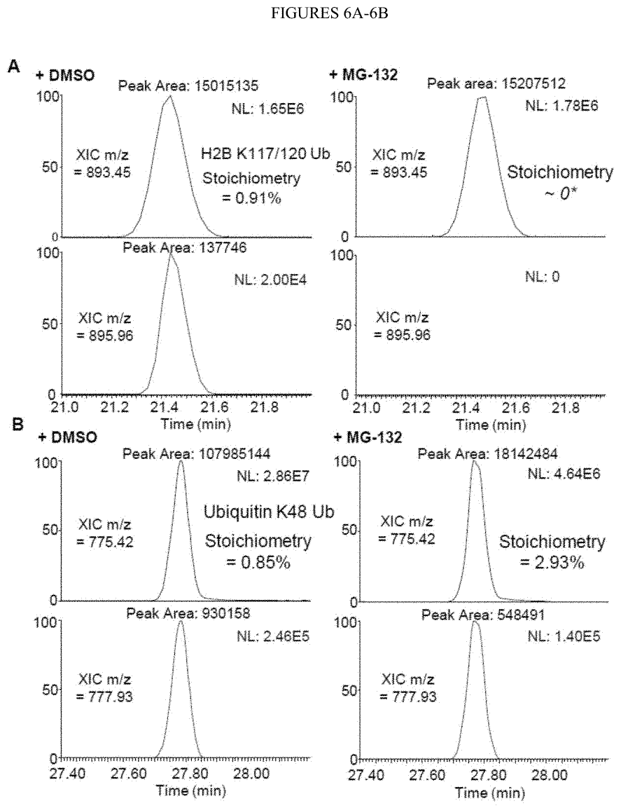

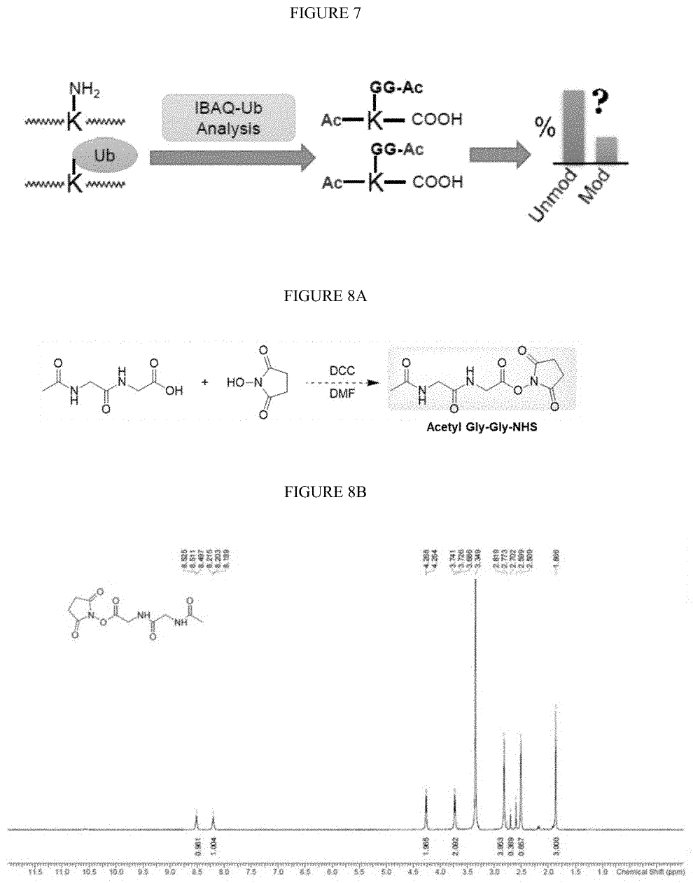

The stoichiometries of known histone Ub sites was also determined. Histone H2B monoubiquitination has been well studied for its roles in gene expression and is required for the subsequent H3K4 and K79 trimethylation in transcriptional activation (Y. Zhang, Genes Dev 2003, 17, 2733-2740). Using traditional biochemical based strategy, it was estimated that a roughly 1% histone H2B in eukaryotic cells carries the modification (M. H. West, W. M. Bonner, Nucleic Acids Res 1980, 8, 4671-4680). Using IBAQ-Ub strategy, the absolute stoichiometry of C-terminal histone H2B ubiquitination was able to be determined by measuring the precursor ion intensities of the peptide containing K117/K120 (E.GTKAVTKYTSSK.-). The analysis showed that histone H2B K117/K120 Ub stoichiometry was 0.91% in 293T cells (FIG. 6A), which is in close agreement with previous estimates and also suggests that C-terminal K117/K120 H2B ubiquitination are the major Ub events on histone H2B. MG132 treatment significantly decreased the abundance of H2B K117/K120 ubiquitination (to a level that was below the detection limit) (FIG. 6B). Such finding actually agreed well with previous observations using orthogonal approaches such as Western blotting or SILAC-based quantitative proteomics (S. A. Wagner, et al., Mol Cell Proteomics 2011, 10, M111 013284; N. D. Udeshi, et al., Mol Cell Proteomics 2013, 12, 825-831; V. Akimov, et al., Nat Struct Mol Biol 2018, 25, 631-640; C. Gao, et al., J Diabetes Res 2013, 2013, 589474; T. Prenzel, et al., Cancer Res 2011, 71, 5739-5753).

The K48 polyubiquitin linkage stoichiometry was further measured on extracted histones. The results showed that K48 linkage stoichiometry was only 0.85% in DMSO treated control samples and it increased significantly to 2.93% upon proteasome inhibition (FIG. 6C-D). Such observation agreed well with the knowledge that K48-linked polyubiquitination leads to proteasome-mediated substrate degradation (D. Komander, M. Rape, Annu Rev Biochem 2012, 81, 203-229; P. Xu, et al., Cell 2009, 137, 133-145). Despite the large increase (more than tripled upon MG132 treatment), the absolute stoichiometry change of K48 ubiquitination on extracted histone proteins was very small, suggesting that the major functional roles of histone ubiquitination are unlikely to target histones for proteasome degradation.

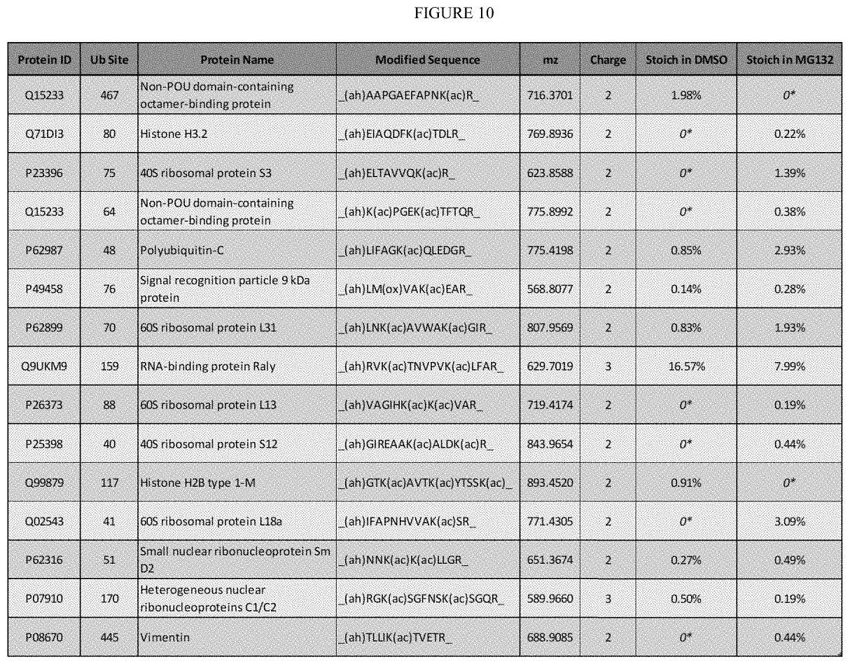

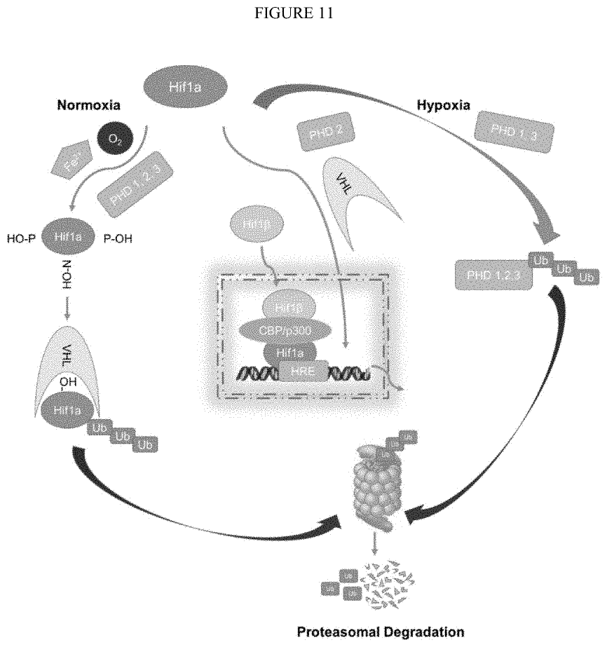

Finally, peptides with known Ub sites were identified in the analysis of histone-enriched chromatin fractions based on the Ub database (www.phosphositeplus.org) and then compared the Ub stoichiometries of sites that were identified in both DMSO and MG132 treated samples (FIG. 10). Overall, MG132 treatment increased the Ub abundance for 72% of the sites that were quantified in both DMSO and MG132 treated samples, while 27% of the sites showed decreased ubiquitination. Interestingly, despite the broad increase in the Ub level upon MG132 treatment, the absolute changes of Ub stoichiometries were very small and the Ub fractional abundance remained very low overall after the proteasome inhibitor treatment. Such data suggested that the inhibition of proteasome activities may have generally limited impact on the absolute levels of site-specific ubiquitination, which may partially explain the lack of correlation between the changes in Ub levels and corresponding protein abundances upon proteasome inhibition observed in a recent study (V. Akimov, et al., Nat Struct Mol Biol 2018, 25, 631-640).

Ubiquitination is a complex posttranslational modification playing vital roles in cellular physiology and protein homeostasis. Unlike small chemical modifications (e.g. acetylation and phosphorylation), it is very difficult to develop antibodies against specific Ub sites. Therefore, studying the dynamics and physiological significance of site-specific ubiquitination using traditional biochemical strategies is challenging. In this study, a chemical proteomics approach (“IBAQ-Ub”) was developed for stoichiometric quantification of site-specific Ub abundance. Comparing to previous approaches, IBAQ-Ub overcomes several challenges. First, it applies NHS-based chemistry for highly efficient and systematic derivatization of amino groups for stoichiometric quantification. Second, incorporation of stable isotopic labeling for quantification eliminates quantification error due to differential LC elution and ionization efficiencies of modified and unmodified peptides and therefore allows accurate quantification. Third, the workflow does not require synthetic peptides or proteins with stable isotope labeling as external standards, reducing the complications during sample preparation. More importantly, comparing to traditional relative quantification of Ub dynamics using SILAC and isobaric tagging, IBAQ-Ub measures the fractional abundance of ubiquitination that reflects the physiological level of the modification. Such measurement allows quantitative comparison of Ub abundances across different sites or substrate proteins. Overall, the chemical-based IBAQ-Ub workflow proposed in this study offers an efficient and generalizable approach to determine the stoichiometric dynamics of ubiquitination at the site-specific level in either targeted or untargeted applications, and therefore, will be a valuable tool for the functional analysis of this important signaling pathway.

### Materials and Methods

### Materials

N-(3-Dimethylaminopropyl)-N-ethylcarbodiimide hydrochloride (EDCl), sodium hydroxide, formic acid, sodium acetate (¹³C₂D₃), Triton X-100, phenylmethylsulfonyl fluoride (PMSF), sodium azide (NaN₃), calcium chloride (CaCl₂), ammonium bicarbonate (NH₄HCO₃), triethylammonium bicarbonate (TEAB), β-mercaptoethanol, penicillin (PCN), streptomycin, L-arginine, and L-Lysine were from Sigma. K48-linked di-Ubiquitin, K48-linked penta-ubiquitin, free ubiquitin were from Bio-techne. N-Hydroxysuccinimide (NHS), acetyl glycylglycine (AcGG), dimethyl sulfoxide (DMSO), water (H₂O), acetonitrile (ACN), phosphate-buffered saline (PBS), hydroxylamine (NH₂OH), guanidine hydrochloride (HNC(NH₂)₂. HCl), urea (CH₄N₂O), tris(2-carboxyethyl) phosphine hydrochloride (TCEP.HCl), hydrochloric acid (HCl), sulfuric acid (H₂SO₄), trifluoroacetic acid (TFA), formic acid (FA), ammonium acetate (NH₄AcO), acetic acid (HAc), tricholoracetic acid (TCA), ethyl alcohol (EtOH), acetone (Ace), dichloromethane (DCM), ninhydrin (C₉H₆O₄), Bradford assays, Dulbecco's modified Eagle medium (DMEM), sodium dodecyl sulfate (SDS), bromophonol blue, and Imperial Protein Stain reagent were from Thermo Fisher. MG-132 was from Pierce. Iodoacetamide and bovine serum albumin (BSA) were from VWR. Trypsin, Asp-N and Glu-C were from Promega. Dialyzed fetal bovine serum (FBS) was from Gibco. ¹³C₆¹⁵N₄-L-arginine and ¹³C₆¹⁵N₂-L-Lysine were from Silantes GmbH. Proteinase inhibitor cocktail was from Roche. Glycerol (C₃H₅(OH)₃) was from Avantor. Tris (Hydroxymethyl) Aminomethane (Tris base) was from RPI.

### Chemical Synthesis

Isotopically labeled acetyl-N-hydroxysuccinimide (or heavy Ac—NHS) was synthesized using sodium acetate (¹³C₂D₅) (Sigma) following the previously described protocol (T. Zhou, et al., J Proteome Res 2016, 15, 1103-1113). Acetyl glycylglycine-N-hydroxysuccinamide (AcGG-NHS) was synthesized through the following procedure. Five mmol of acetyl glycylglycine was added to 7.75 mmol of N-hydroxysuccinimide in N-methyl-2-pyrrolidone (NMP), and to this mixture was added 7.75 mol of N,N′-dicyclohexylcarbodiimide (DCC) in NMP at −20° C. The reaction was stirred at 15° C. for 4 hours, and was subsequently cooled to 0° C. and filtered. The filtrate was added to 400 ml of methyl tert-butyl ether (MTBE), which produced a thin film of yellow oil. After complete layer formation, the MTBE layer was discarded and the oil was added to acetonitrile and lyophilized to obtain the resultant AcGG-NHS compound. The purity of the compound (˜90%) was confirmed by HPLC analysis and the structure of the compound was confirmed by ¹H NMR (400 MHz, DMSO-d₆) δ 8.51 (t, J=5.7 Hz, 1H), 8.20 (t, J=5.3 Hz, 1H), 4.26 (d, J=5.8 Hz, 2H), 3.73 (d, J=6.0 Hz, 2H), 2.82 (s, 4H), 1.87 (s, 3H).

### Ninhydrin Test

To test the amine labeling efficiency of AcGG-NHS, three groups of samples were prepared—Blank: 100 μL of 9 M urea/1×PBS, Control: 0.1 mg BSA (5 μL of 20 mg/mL BSA) and 95 μL of 9 M urea/1×PBS, Test: 0.1 mg BSA (5 μL of 20 mg/mL BSA) and 95 μL of 9 M urea/1×PBS. First, samples in each groups were reduced with a final concentration of 5 mM TCEP and alkylated with a final concentration of 5 mM iodoacetamide at room temperature with shaking in dark for 30 min. Then, samples in the Test group were derivatized with AcGG-NHS following the procedure described below. Each group was followed by a reaction with ⅕ the volume of Ninhydrin reagent solution (0.35 g ninhydrin in 100 mL EtOH) at 85° C. with gentle stirring for 5 min. Absorbance of each sample was measured at 577 nm using NanoDrop spectrophotometer (Fisher).

### SILAC Cell Culture and AcGG-NHS Reactivity Test

HT22 cells were cultured in DMEM medium for SILAC, which was supplemented with L-arginine and L-lysine (light AA) or ¹³C₆¹⁵N₄-L-arginine and ¹³C₆¹⁵N₂-L-lysine (heavy AA), as previously described (J. Park, et al., Mol Cell 2013, 50, 919-930). The cells were labeled for a minimum of 6 generations and lysed in 9M urea/PBS with protease inhibitor cocktail, followed by reduction/alkylation with 5 mM TCEP and iodoacetamide. The labeling efficiency of heavy labeled cells were confirmed by MS analysis to be ˜99%. To quantify the reactivity of AcGG-NHS, the heavy AA labeled proteins were derivatized with AcGG-NHS as described below and then mixed with an equal amount of light AA labeled proteins. The mixed cell lysate was diluted 6 folds with PBS and digested by trypsin for LCMS analysis.

### Histone Extraction

293T cells were treated with 5 μM MG132 or the equal volume of DMSO for 12 hours. Histone extraction was performed as described previously (D. Shechter, et al., Nat Protoc 2007, 2, 1445-1457). Briefly, the cells were harvested by trypsin digestion. The cell pellets were washed with cold PBS and then lysed in 0.5 mL Extraction Buffer (PBS+0.5% Triton X-100 (v/v)) with protease inhibitor cocktail. After centrifuge, the supernatant was discarded and the pellet was washed with Extraction Buffer again to remove residual supernatant solution in pellet. The pellet was re-suspended in 0.4 ml 0.4 N H₂SO₄ for acid extraction. After overnight incubation at 4° C., the supernatant was extracted and proteins were precipitated with a final concentration of 20% TCA (v/v), followed by cold acetone wash. The protein pellet was air dried and protein concentration was measured by the Bradford assay after solubilizing the pellet with water.

### AcGG-NHS Labeling and IBAQ Analysis Workflow

Ten μg of proteins were dried in a Speed-Vac and resuspended in 10 μL of 9 M urea/PBS with pH adjusted to ˜8.5. AcGG-NHS stock solution was prepared by dissolving AcGG-NHS in 50% ACN/50% H₂O mixture at a concentration of 100 mg/mL. One μL of AcGG-NHS stock solution was added to each sample and the mixture was vortexed for 45 min. The labeling was repeated for another two times. The reaction was quenched with 5% hydroxylamine (pH=6.0, 1.5 M) for 15 min. Each sample was diluted 6× with PBS and proteins were digested with trypsin for overnight at 37° C. (enzyme-to-substrate ratio of 1:100 (w/w)). When a second digestion was needed, another protease (Glu-C or Asp-N) was added (enzyme to substrate ratio of 1:100 w/w) for overnight digestion at 37° C. Following digestion, peptides were desalted and then resuspended in 10 μL of 9M urea/PBS at a pH ˜8.5. Heavy Ac—NHS stock solution was prepared by dissolving heavy Ac—NHS in ACN at a concentration of 100 mg/mL. One μL of heavy Ac—NHS stock solution was added to each sample for 45-min labeling. The labeling was repeated for two more times and the reaction was quenched with 5% hydroxylamine (pH=6.0, 1.5 M) for 15 min. The peptides were desalted with stage tips (C₁₈) prior to LCMS analysis (J. Rappsilber, et al., Nat Protoc 2007, 2, 1896-1906).

To check the labeling efficiency of the AcGG-NHS, the total intensity of peptides with AcGG (K) labeling was compared to that of peptides with heavy Ac (K) or with unmodified lysine. To check the labeling efficiency of heavy Ac on peptide N-terminus, the total intensity of peptides with blocked N-terminus was compared to that of peptides with free N-terminus or with underivatized GG remnant.

### IRAQ Analysis of Penta-Ub

Ten μg of commercial K48-linked penta-Ubiquitin (Bio-techne) was dissolved in 9M urea/PBS buffer and subject to in-solution AcGG-NHS labeling as described above. Upon completion of quenching, an equal volume of 2× sample SDS buffer (1 M Tris-HCl pH 6.8, 10% SDS, glycerol, 1% bromophonol blue, β-mercaptoethanol) was added to the sample for 15 min incubation at 37° C. Then the proteins were separated by SDS/PAGE followed by staining with colloidal coomassie blue. The gel band corresponding to penta-Ubiquitin was cut out and the protein was in-gel digested by trypsin following a previously described protocol (A. Shevchenko et al., Nat Protoc 2006, 1, 2856-2860). The extracted peptides were resuspended in 104 of 9 M urea/PBS and subjected to heavy Ac—NHS labeling. Peptides were then desalted by C₁₈ Stage-tip prior to LCMS analysis.

### HPLC-MS/MS Analysis

Mass spectrometry data were collected using an Orbitrap Fusion mass spectrometer (Thermo Scientific) with a Top-12 method. Samples were resolubilized in HPLC buffer A (0.1% formic acid in water, v/v) and injected onto a self-packed reversed-phase capillary HPLC column (15 cm×100 μm, ReproSil-Pur Basic C18, 2.5 μm, Dr. Maisch GmbH). Peptides were separated by a Proxeon Easy nLC 1000 Nano-UPLC system (Thermo Scientific) at a flow rate of 200 nL/min with a 56 min gradient of 7-32% HPLC buffer B (0.1% formic acid in acetonitrile, v/v). The Orbitrap Fusion mass spectrometer (Thermo Scientific) was operated in a positive polarity mode with ion transfer tube temp of 275° C. Precursor ions were acquired in Orbitrap with a resolution 60,000 at 200 m/z and a mass range of 380-1800 m/z. Dynamic exclusion was enabled with an exclusion duration of 15 s and a mass tolerance of ±25 ppm. Fragment ions were acquired in the linear ion trap with an isolation window of 1.6 m/z and high energy collisional dissociation (HCD) of 35%.

### Sequence Database Searching and Data Processing

Mass spectra were processed with MaxQuant software (version 1.5.3.12) (J. Cox, M. Mann, Nat Biotechnol 2008, 26, 1367-1372). Peptides were identified using the integrated Andromeda search engine (J. Cox, et al., J Proteome Res 2011, 10, 1794-1805) with default settings against extracted human histone database or UniProt databases for human or mouse at a 1% false discovery rate (FDR). Carbamidomethylation of cysteine residues was set as a fixed modification, whereas acetylation of protein N-termini, AcGG labeling of protein N-termini and lysine, heavy Ac labeling of peptide N-termini were specified as variable modifications. To determine the stoichiometry of known ubiquitination sites in the histone-enriched protein fraction, the identified AcGG-labeled peptides were first compared with the publically available ubiquitination database (www.phosphosite.org) (P. V. Hornbeck, et al., Nucleic Acids Res 2015, 43, D512-520) to identify peptides with known ubiquitination sites. Manual evaluation was then applied to analyze the extracted ion chromatograms of the identified peptides and corresponding heavy AcGG labeled peptides. Ubiquitination stoichiometry was calculated by the peak area of the heavy AcGG peptide divided by the sum of light and heavy AcGG labeled peptides.

### Example 2. Characterization of the Ubiquitination Signaling on Hypoxia-Inducible Factor with Quantitative Chemical Proteomic Analysis

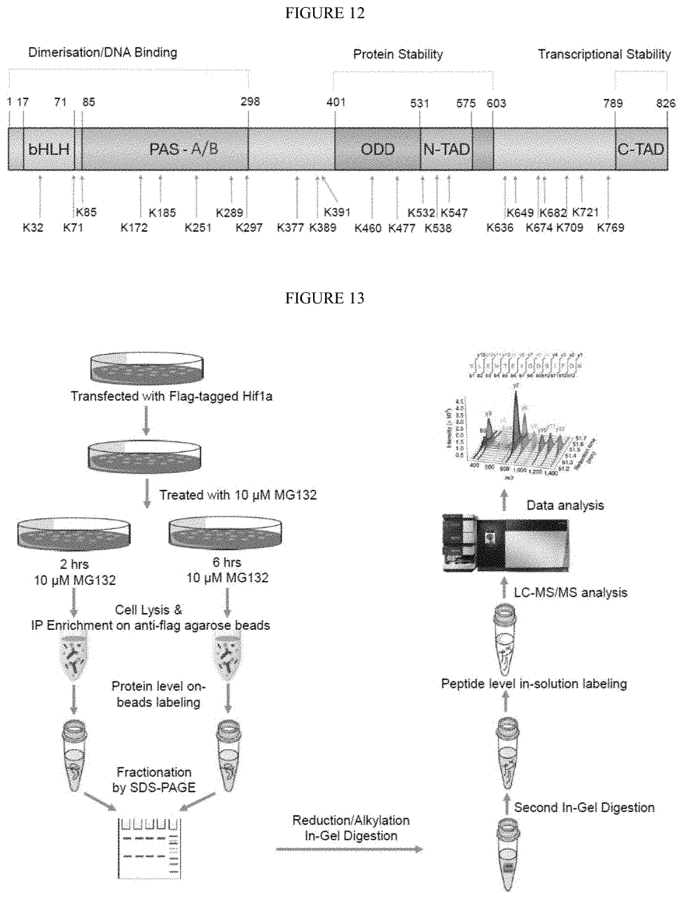

Hypoxia inducible factors (HIF-alpha) are key regulators of cellular oxygen-sensing pathways. Under normoxia, HIF-alpha proteins are poly-ubiquitinated and degraded through a proteasome-dependent mechanism (Majmundar, et al., Mol Cell 40, 294-309 (2010)) (see, FIG. 11) Ubiquitination of HIF-alpha proteins is mediated by proline hydroxylation-dependent interaction with E3 ubiquitin ligase pVHL (Pugh, et al., Semin Cancer Biol 13, 83-89 (2003)). HIF1A is one of the HIF-alpha family proteins. Under hypoxia, HIF1A promotes the activation of diverse hypoxia response pathways involved in cellular metabolism and angiogenesis. Advances in proteomics analysis has revealed that HIF1A is an extensively ubiquitinated protein (Akimov, V. et al. Nat Struct Mol Biol 25, 631-640 (2018)) and yet, the abundance and regulation of HIF1A ubiquitination at the site-specific level remain poorly characterized (see, FIG. 12).

In this study, the IBAQ-Ub workflow (see, Li, Y. et al. Angew Chem Int Ed Engl 58, 537-541 (2019), which is incorporated by reference herein for all purposes), was used to quantify the prevalence and dynamics of ubiquitination signaling on HIF1A under different cellular environments. Aspects of the IBAQ-Ub workflow are described in Example 1 and shown in FIGS. 1A-1C. In brief, ubiquitin is conjugated to the target lysine residue via side-chain isopeptide bond. Upon trypsin cleavage, a GG dipeptidyl structure remains conjugated to the ubiquitinated lysine and serves as a mark for proteomic identification of ubiquitination site. To allow intensity-based site-specific stoichiometry analysis of ubiquitination, a GG remnant homologue with acetylated N-terminal (AcGG tag) was developed (see, FIG. 1B). As shown in FIG. 1C, the IBAQ-Ub workflow involves the first step labeling of unmodified lysines with AcGG tag, tryptic cleavage of proteins into peptides and second labeling of peptides with heavy acetyl group (13C2D3-NHS,*Ac) (Zhou, T., et al., J Proteome Res, 2016. 15(3): p. 1103-13) to distinguish previously unmodified lysine from previously ubiquitinated lysine and allow MS-intensity-based stoichiometry analysis of Ub

### Methods

Flag-tagged HIF1A was expressed in 293T cells. The cells were treated with 10 μM MG132 for 2 hr and 6 hr respectively. HIF1A was purified by immunoprecipitation with antibody against the Flag epitope. Stoichiometric analysis of purified endogenous HIF1A was performed with a modified IBAQ-Ub workflow. Proteins were labeled with AcGG-NHS and then resolved on SDS-PAGE. Following in-gel digestion, peptides were labeled with heavy acetyl NHS (Zhou, T., et al., J Proteome Res, 2016. 15(3): p. 1103-13) prior to be analyzed by HPLC-MS/MS.

### Results

For stoichiometric analysis of ubiquitination on a single protein target, we adapted the traditional immunoprecipitation protocol and optimized IBAQ-Ub workflow with on-beads AcGG-NHS labeling, in-gel digestion and heavy acetyl-NHS labeling. SDS-PAGE and in-gel digestion efficiently removed hydroxylamine used in the AcGG labeling as a quenching reagent and enabled efficient secondary labeling with heavy Ac—NHS. Quantitative analysis of ubiquitin linkage revealed the temporal dynamic activation of polyubiquitination on HIF1A in response to proteasome inhibition. Site-specific analysis of HIF1A ubiquitination dynamics identified most of the known ubiquitination sites of HIF1A, corroborating this IBAQ-Ub workflow. The analysis identified endogenous dynamics of ubiquitination sites on HIF1A that shows early or late response to proteasome inhibition as well as predominant ubiquitination sites in response to the treatment. Further chemical or siRNA based treatment analysis may be used to identify major ubiquitination targets of E3 ligase such as Cullin-VHL or deubiquitinase.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method of quantifying ubiquitin-like modification in a test protein sample comprising:
a) contacting the test protein sample with a compound of formula (I): 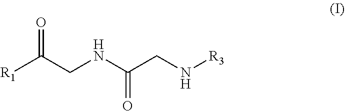
to provide a first labeled test protein sample, wherein R₁ together with the carbonyl group to which it is attached forms a group capable of reacting with an amino group to form an amide; and R₃ is X—Y—, wherein X is (C₁-C₆)alkanoyl and Y is absent or arginine;
b) contacting the first labeled test protein sample with a first enzyme, wherein the first enzyme cleaves ubiquitin-like protein(s) from all modified amino acid residues, to provide a second labeled test protein sample;
c) contacting the second labeled test protein sample with a compound of formula (II): 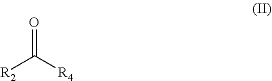
to provide a third labeled test protein sample, wherein R₂ together with the carbonyl group to which it is attached forms a group capable of reacting with an amino group to form an amide, and wherein R₄ is (C₁-C₅)alkyl; and
d) measuring the molecular weight of the protein(s) in the third labeled test protein sample to quantify the ubiquitin-like modification(s) in the test protein sample,
wherein the compound of formula I is isotopically labeled; the compound of formula II is isotopically labeled; or the compound of formula I and the compound of formula II are differentially isotopically labeled.

2. The method of claim 1, further comprising comparing the molecular weight of the protein(s) in the third labeled test protein sample to the molecular weight of a corresponding control protein(s), to quantify the ubiquitin-like modification(s) in the test protein sample.

3. The method of claim 1, wherein the ubiquitin-like modification is ubiquitination, SUMOylation, ISGylation or neddylation.

4. The method of claim 1, wherein R₁ is:

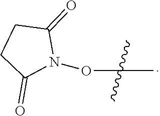 [SMILES: *ON1C(=O)CCC1=O]

5. The method of claim 1, wherein R₁ is:

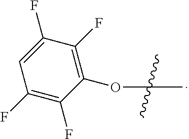 [SMILES: *Oc1c(F)c(F)cc(F)c1F]

6. The method of claim 1, wherein X is —C(═O)CH₃ or —C(═O)CH₂CH₃.

7. The method of claim 1, wherein Y is absent.

8. The method of claim 1, wherein Y is arginine.

9. The method of claim 1, wherein the compound of formula (I) is:

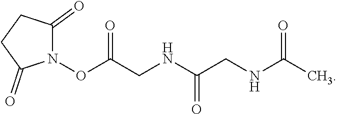 [SMILES: CC(=O)NCC(=O)NCC(=O)ON1C(=O)CCC1=O]

10. The method of claim 1, wherein the compound of formula (I) is:

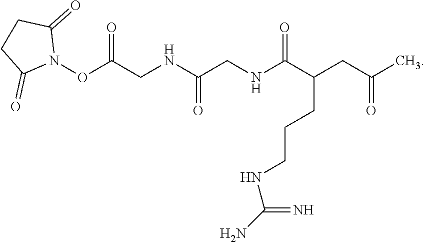 [SMILES: CC(=O)CC(CCCNC(=N)N)C(=O)NCC(=O)NCC(=O)ON1C(=O)CCC1=O]

11. The method of claim 1, wherein the compound of formula (I) is isotopically labeled.

12. The method of claim 1, wherein the first enzyme is trypsin, Arg-C or WaLP.

13. The method of claim 1, wherein R₂ is:

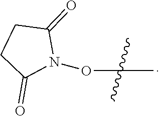 [SMILES: *ON1C(=O)CCC1=O]

14. The method of claim 1, wherein R₂ is:

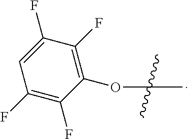 [SMILES: *Oc1c(F)c(F)cc(F)c1F]

15. The method of claim 1, wherein R₄ is C₁(alkyl).

16. The method of claim 1, wherein R₄ is C₂(alkyl).

17. The method of claim 1, wherein the compound of formula (II) is isotopically labeled.

18. The method of claim 1, further comprising contacting the second labeled test protein sample with a second enzyme selected from the group consisting of Glu-C, Asp-N, chymotrypsin, pepsin, aminopeptidase, carboxypeptidase, elastase, thermolysin and TEV protease, wherein the second enzyme digests one or more proteins in the second labeled test protein sample to provide a mixture of 2 or more peptide fragments.

19. The method of claim 1, wherein the molecular weight of the protein(s) in the third labeled test protein sample is measured using mass spectrometry or liquid chromatography-mass spectrometry (LC-MS).

20. A method of screening a test compound for modulating activity of ubiquitin-like modification, the method comprising:
a1) contacting a test protein sample with a test compound to provide a test protein reaction sample;
a2) contacting the test protein reaction sample with a compound of formula (I): 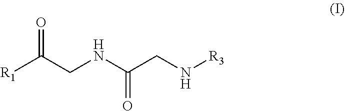
to provide a first labeled test protein reaction sample, wherein R₁ together with the carbonyl group to which it is attached forms a group capable of reacting with an amino group to form an amide; and R₃ is X—Y—, wherein X is (C₁-C₆)alkanoyl and Y is absent or arginine;
b) contacting the first labeled test protein reaction sample with a first enzyme, wherein the first enzyme cleaves ubiquitin-like proteins from all modified amino acid residues, to provide a second labeled test protein reaction sample;
c) contacting the second labeled test protein reaction sample with a compound of formula 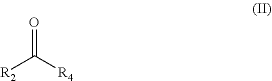
to provide a third labeled test protein reaction sample, wherein R₂ together with the carbonyl group to which it is attached forms a group capable of reacting with an amino group to form an amide, and wherein R₄ is (C₁-C₅)alkyl;
d) measuring the molecular weight of the protein(s) in the third labeled test protein reaction sample to quantify the ubiquitin-like modification(s) in the test protein sample,
wherein the compound of formula I is isotopically labeled; the compound of formula II is isotopically labeled; or the compound of formula I and the compound of formula II are differentially isotopically labeled; and
e) identifying the test compound as having modulating activity of ubiquitin-like modification when the amount or location of ubiquitin-like modification in the test protein sample is different than the ubiquitin-like modification in a corresponding control protein sample.

21. A method of identifying a subject having a disease or disorder associated with altered ubiquitin-like modification, the method comprising:
a) contacting a test protein sample from the subject with a compound of formula (I): 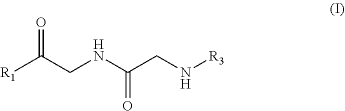
to provide a first labeled test protein sample, wherein R₁ together with the carbonyl group to which it is attached forms a group capable of reacting with an amino group to form an amide; and R₃ is X—Y—, wherein X is (C₁-C₆)alkanoyl and Y is absent or arginine;
b) contacting the first labeled test protein sample with a first enzyme, wherein the first enzyme cleaves ubiquitin-like protein(s) from all modified amino acid residues, to provide a second labeled test protein sample;
c) contacting the second labeled test protein sample with a compound of formula (II): 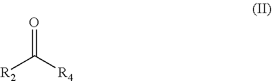
to provide a third labeled test protein sample, wherein R₂ together with the carbonyl group to which it is attached forms a group capable of reacting with an amino group to form an amide, and wherein R₄ is (C₁-C₅)alkyl;
d) measuring the molecular weight of the protein(s) in the third labeled test protein sample to quantify the ubiquitin-like modification(s) in the test protein sample,
wherein the compound of formula I is isotopically labeled; the compound of formula II is isotopically labeled; or the compound of formula I and the compound of formula II are differentially isotopically labeled; and
e) identifying the subject as having a disease or disorder associated with altered ubiquitin-like modification when the amount or location of modification(s) in the test protein sample is different than the modification(s) in a corresponding control protein sample.

22. The method of claim 21, wherein the disease or disorder associated with altered ubiquitin-like modification is cancer, a neurodegenerative disease, cystic fibrosis, muscle wasting or an immunological disorder.

23. A kit for quantifying ubiquitin-like modification in a test protein sample, the kit comprising:
1) a compound of formula (I): 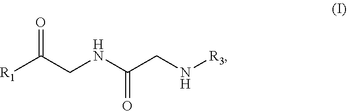
wherein R₁ together with the carbonyl group to which it is attached forms a group capable of reacting with an amino group to form an amide; and R₃ is X—Y—, wherein X is (C₁-C₆)alkanoyl and Y is absent or arginine;
2) a compound of formula (II): 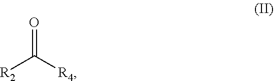
wherein R₂ together with the carbonyl group to which it is attached forms a group capable of reacting with an amino group to form an amide, and wherein R₄ is (C₁-C₅)alkyl; and
3) instructions for quantifying ubiquitin-like modification in the test protein sample,
wherein the compound of formula I is isotopically labeled; the compound of formula II is isotopically labeled; or the compound of formula I and the compound of formula II are differentially isotopically labeled.

24. A compound of formula (Ia): 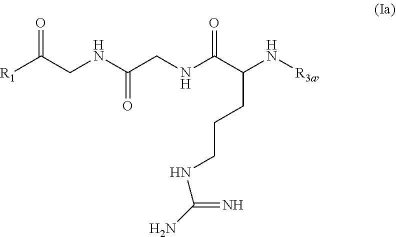
wherein R₁ is an activating group capable reacting with an amino group to form an amide; and R₃ₐ is (C₁-C₆)alkanoyl.
